(19) United States
(12) Patent Application Publication
(10) Pub. No.: US 2020/0009222 A1
(43) Pub. Date: Jan. 9, 2020

(54) THERAPEUTICALLY ACTIVE COMPLEXES

(71) Applicant: HAMLET PHARMA AB, Lund (SE)

(72) Inventors: Catharina SVANBORG, Malmo (SE); Aftab NADEEM, Lund (SE); Kenneth Hun MOK, Dublin (IE); Chin Shing HO, Lund (SE)

(21) Appl. No.: 16/468,605

(22) PCT Filed: Dec. 19, 2017
(86) PCT No.: PCT/IB2017/058140

§ 371 Date: Jun. 11, 2019

(30) Foreign Application Priority Data
Dec. 20, 2016 (GB) 1621752.3

(51) Int. Cl. A61K 38/17; A61K 38/46; A61K 47/54; A61P 35/00

(52) U.S. Cl. CPC A61K 38/1709; A61P 35/00; A61K 47/542; A61K 38/46

(57) ABSTRACT

A biologically active complex comprising a peptide of up to 50 amino acids in length which comprises an alpha-helical domain of a protein which has membrane perturbing activity or a variant thereof which lacks cysteine residues, and oleic acid or a salt thereof, provided the protein is other than alpha-lactalbumin. Complexes of this type are useful in therapy, in particular cancer therapy.

The present invention relates to a class of peptides which have therapeutic activity, in particular as anti-cancer or anti-tumour agents. Methods for preparing these peptides, as well as pharmaceutical compositions containing them form a further aspect of the invention.

### BACKGROUND TO THE INVENTION

Novel cancer treatments should ideally combine efficacy with selectivity for the targeted tumor and new, targeted therapies act with greater precision. Tissue toxicity and side effects are still the norm, however, and the notion of new, tumor specific mechanisms of cell death is justly regarded with skepticism. Yet, recent investigations into the tumoricidal effects of certain protein-lipid complexes suggest that tumor cells may share conserved mechanisms of cell death that distinguish them from normal, differentiated cells. These protein-lipid complexes insert into lipid bilayers and trigger cell death by perturbing the membrane structure of tumor cells. The subsequent internalization and inhibition of critical cellular functions distinguishes tumor cells from healthy differentiated cells and as a result, the tumor cells are killed while normal, differentiated cells survive.

These properties identify protein-lipid complexes as interesting drug candidates, with broad tumoricidal activity and documented tumor specificity. The feasibility of this approach is illustrated by HAMLET (Human Alpha-lactalbumin Made LEthal to Tumor cells), which was discovered serendipitously, in a fraction of human milk. HAMLET kills many different tumor cells with rapid kinetics and shows therapeutic efficacy in animal models of colon cancer, glioblastoma and bladder cancer. Investigator-driven clinical trials have demonstrated that HAMLET is active topically, against skin papillomas and induces shedding of dead tumor cells into the urine of patients with bladder cancer.

Alpha-lactalbumin is the most abundant protein in human milk, essential for the survival of lactating mammals, due to its role as a substrate specifier in the lactose synthase complex. HAMLET is formed by partial unfolding of globular alpha-lactalbumin and binding of deprotonated oleic acid, with a stoichiometry of ¼-8.

A number of peptides derived from alpha-lactalbumin have also been found to have therapeutic effects in their own right (see for example WO2012/069836).

### SUMMARY OF THE INVENTION

The applicants have identified specific alpha-lactalbumin peptide domains as the functional ligands for tumor cell recognition and death. Shared peptide reactivity among tumor cells from different tissues suggests that the alpha-helical peptide is recognized by tumor cell membranes in the context of oleic acid and that this interaction triggers a conserved death response in cancer cells and established cancers, in vivo. Thus, complexes comprising these peptides show broad tumoricidal activity, as exemplified by work done with the known complexes based upon alpha-helical domains of alpha-lactalbumin. However, the applicants have surprising found that these effects can be generalized to other alpha-domain peptides with membrane perturbing activity.

This work identifies a new, general mechanism by which alpha-helical peptides can target and kill tumor cells. The applicants have found that membrane interactive peptide-domains form oleate complexes with broad tumoricidal activity. This concept is exemplified by the N-terminal alpha helices of alpha-lactalbumin, which invades tumor cells and accumulates in nuclear speckles, where it suppresses transcription through a direct effect on the speckle constituents SC-35, PKC and Pol II. This “gain of function” was reproduced for a range of alpha peptides having a wide variety of sequences including Sar1 in the COPII family, where the alpha-helical, membrane-integrating peptide gained tumoricidal activity, when mixed with oleate. The beta sheet domains of these proteins, in contrast, were sorted to the lysosomes for degradation. Synthetic alpha-1 peptide formed therapeutic oleate complexes that reduced tumor load in a murine bladder cancer model. These findings suggest that tumor cells recognize alpha-helical peptide motifs in the context of oleate and respond by activating a conserved mechanism of tumor cell death.

According to the present invention, there is provided a biologically active complex comprising a peptide of up to 50 amino acids in length which comprises an alpha-helical domain of a protein which has membrane perturbing activity or a variant thereof which lacks cysteine residues, and oleic acid or a salt thereof, provided the protein is other than alpha-lactalbumin. The finding that the precise sequence of the peptide is not critical and but rather it is the inclusion of an alpha-helical domain generally is sufficient to provide active complexes is unexpected.

As used herein the expression ‘alpha-helical domain’ refers to a motif in the secondary structure of the peptide in which a right-hand coiled or spiral conformation (helix) is formed, in which every backbone N—H group donates a hydrogen bond to the backbone C═O group of the amino acid located three or four residues earlier along the peptide sequence.

In particular, the alpha-helical domain forms an N-terminal domain within the membrane-perturbing protein.

Suitably the peptide is up to 40 amino acids in length, for example up to 30 amino acids, or up to 25 amino acids in length. Typically, the peptide will be from 20-40 amino acids in length.

Suitable peptides will comprise fragments of membrane perturbing proteins. These are proteins which have the capability of interacting with the interface of cell membranes, in particular causing disruption such as tubulation of the cell membrane. Typically, the protein will become embedded in the cell membrane. Thus the protein will comprise a transmembrane domain and/or a membrane binding region, and such domains or regions may be at least partially included in the peptides of the invention. Examples of such proteins include coat complexes such as COPI, COPII (such as SAR 1), HOPS/CORVET, SEA (Seh1-associated), and clathrin complexes BAR domain proteins such as endophilins, and the ESCRT complex, including Snf7 domain subunits.

In a particular embodiment, the peptide is derived from a COPII family protein such as SAR1. A particular example of such a peptide is a peptide of SEQ ID NO 4

(SEQ ID NO 4)
MAGWDIFGWF RDVLASLGLW NKH

The alpha-helical domain of said proteins would be well understood in the art, or may be determined using conventional methods.

In another particular embodiment, the peptide is derived from an endophilin peptide, in particular an N-terminal peptide. A particular example of such a peptide is a Endophilin-Res1-35, which is a peptide of SEQ ID NO 5:

(SEQ ID NO 5)
MSVAGLKKQF HKATQKVSEK VGGAEGTKLD DDFKE

Other specific examples of peptides which are derived from transporter proteins such as ABC or ion transporter proteins.

Thus particular examples of such peptides include:

(SEQ ID NO 6)
9-SF SSLGLWASGL ILVLGFLKLI HLLLRRQT-38 (Lung -
CYP4B1)
(SEQ ID NO 7)
15-SEKKK TRRCNGFKMF LAALSFSYIA KALG-34 (Liver-
SLCO1B3)
(SEQ ID NO 8)
96-GTPEYVKFAR QLAGGLQCLMWVAAAICLIA-125 (Stomach -
ATP4A)
(SEQ ID NO (9)
67-VQIPYEVTLW ILLASLAKIG FHLYHRLPG-95 (Stomach -
SLC9A4)

Where the alpha-helical domain contains a cysteine residue, these may, in some embodiments, be modified to a different amino acid residue, such as an alanine residue, in order to avoid inter-molecular disulphide bonds.

Thus, in a particular embodiment, the peptide used in the complex of the invention is selected from the group consisting of:

a variant of SEQ ID NO 7, of SEQ ID NO 10:
(SEQ ID NO 10)
SEKKKTRRANGFKMFLAALSFSYIAKALG;
or
a variant of SEQ ID NO 8, of SEQ ID NO 11:
(SEQ ID NO 11)
GTPEYVKFARQLAGGLQALMWVAAAIALIA.

The complex will further comprise oleic acid or a salt thereof In particular, the complex further comprises a water soluble oleate salt. Particular examples of suitable salts may include alkali or alkaline earth metal salts. In a particular embodiment, the salt is an alkali metal salt such as a sodium- or potassium salt.

According to a further aspect of the present invention there is provided a method for preparing a biologically active complex as described above. Said method may comprise combining together peptide as defined above; with oleic acid or a salt thereof, under conditions in which they form a biologically active complex.

Typically, the preparation may be carried out simply by mixing together a suitable peptide and oleic acid or a salt thereof, for example in a solution such as an aqueous solution. The ratio of oleate: peptide added to the mixture is suitably in the range of from 20:1 to 1 to 1, but preferably an excess of oleate is present, for instance in a ratio of oleate:peptide of about 5:1. The mixing can be carried out at a temperature of from 0-50° C., conveniently at ambient temperature and pressure. This simple preparation method provides a particular advantage for the use of such peptides in the complexes. The methods can be carried out in situ, when required for treatment. Thus kits may be provided comprising peptides and salts for mixing immediately prior to administration.

Such kits, and reagents for use in the kits form a further aspect of the invention.

Peptides are suitably synthetic peptides although they may be prepared by recombinant DNA technology.

Peptides useful in forming the complexes of the invention may be novel and these form yet a further aspect of the invention.

The complex of the invention can be used in the treatment of cancer. For this purpose, the complex is suitably formulated as a pharmaceutical composition.

Thus, complexes as described above and/or oleate salts also as described above, may be formulated into useful pharmaceutical compositions by combining them with pharmaceutically acceptable carriers in the conventional manner. Such compositions form a further aspect of the invention.

The compositions in accordance with this aspect of invention are suitably pharmaceutical compositions in a form suitable for topical use, for example as creams, ointments, gels, or aqueous or oily solutions or suspensions. These may include the commonly known carriers, fillers and/or expedients, which are pharmaceutically acceptable.

Topical solutions or creams suitably contain an emulsifying agent for the protein complex together with a diluent or cream base.

The daily dose of the complex varies and is dependent on the patient, the nature of the condition being treated etc. in accordance with normal clinical practice. As a general rule from 2 to 200 mg/dose of the biologically active complex is used for each administration.

In a further aspect of the invention, there is provided a method for treating cancer which comprises administering to a patient in need thereof, a biologically active complex as described above.

In particular, the complex may be used to treat cancers such as human skin papillomas, human bladder cancer, kidney cancer, lung cancer and glioblastomas. In the latter case, administration may be by infusion as is known in the art.

The invention further provides the biologically active complex as defined above for use in therapy, in particular in the treatment of cancer.

Sensing of the extracellular milieu is essential for cellular life. Membranes are organized to distinguish “outside from inside” and to protect the cell interior from harm, inflicted by changes of the extracellular environment. As a consequence, the sampling of extracellular molecules is tightly regulated, through the expression of receptors and specific signaling pathways, controlling key functional modules, such as proliferation, metabolism and apoptosis. In addition, lipid membranes can sense external ligands, unaided by specific receptors. A series of physicochemical parameters control membrane integrity and signals to the cell interior may be generated by morphological or compositional changes affecting membrane integrity. Proteins that affect membrane curvature are often enriched for alpha-helical structure, and their insertion into spherical lipid membranes may cause tubulation and recruit cytosolic factors to newly created membrane compartments (see for example Barlowe, C. et al. Cell 77, 895-907 (1994); Stagg, S. M. et al. Cell 134, 474-484, doi:10.1016/j.cell.2008.06.024 (2008); Shimada, A. et al. Cell 129, 761-772, doi:10.1016/j.cell.2007.03.040 (2007); Field, M. C. et al., The Journal of cell biology 193, 963-972, doi:10.1083/jcb.201102042 (2011); Frost, A. et al., Cell 137, 191-196, doi:10.1016/j.cell.2009.04.010 (2009); McMahon, H. T. et al., Nature 438, 590-596, doi:10.1038/nature04396 (2005); and Schekman, R. et al. Science (New York, N.Y.) 271, 1526-1533 (1996).

The applicants have demonstrated a new role for membrane-integrating, alpha-helical peptides as cancer therapeutics. As illustrated hereinafter, the N-terminal alpha-helices of alpha-lactalbumin wherein cysteine residues had been replaced by alanines, and Sar1 both gained tumoricidal activity, after forming complexes with oleate; the deprotonated form of oleic acid. Loss of tumor cell viability was demonstrated in vitro for cancer cells from the lung, kidney and urinary bladder and in vivo in a murine bladder cancer model.

Without being bound by theory, this effect appears to be defined by the secondary structure of the peptides and specifically by the alpha-helical confirmations. Partial unfolding of alpha lactalbumin is required to expose the alpha-helical domains and a more flexible conformation is presumably inherent to short peptides, as long-range native contacts usually are absent, thus mimicking partially unfolded states. The results therefore suggest that alpha-helical peptide domains such as the alpha1 and Sar1-alpha23, may gain activity based on the flexible and slightly chaotic tertiary structure of the entire protein and ability to bind relevant cofactors such as lipids. Due to this post-fatty-acid-binding, gain-of-function property, these peptides are clearly distinguished from the antimicrobial peptides (AMP) or alpha-helical membrane-active peptides that do not exhibit relatively selective tumoricidal properties. This “gain-of-function” strategy may be essential to provide more tissue-specific solutions to challenges such as tumor development.

The accumulation in nuclear speckles was visible as a “string of pearls” in the nuclear periphery and further defined by co-localization of the alpha-domain peptides with SC-35 in this structure. The applicants also detected direct effects on nuclear speckle constituents and gene expression was inhibited. Nuclear speckles are important sub-nuclear compartments, which work in concert to coordinate gene expression, including transcription, pre-mRNA processing and mRNA transport. Transcriptionally active genes localize to the speckles, where a continuous and rapid molecular exchange takes place with the surrounding nucleoplasm. According to a model of stochastic self-organization, “high-affinity interactions help to establish a steady-state residency time within these domains”. It seems possible that HAMLET, as well as the complexes of the present invention, may disturb this stochastic self-organization, by establishing high affinity complexes with histone H3, which damage the architecture of the transcriptional machinery and prevent the dissociation of bound components. These effects were further defined by the alpha-helical peptides through a direct effect on PKC dependent phosphorylation of SC-35, which in turn, inhibits Pol II activation. The resulting inhibition of gene expression, which involved H3 and proteasome-centric gene networks, may mark a “point of no return” for the dying tumor cell, as de novo synthesis of critical constituents is severely impaired.

Transitional cell carcinomas are common urological malignancies, with severe consequences, due to a high recurrence rate and lack of curative therapies. Tumors confined to the mucosa are often treated by transuretheral resection, followed by intravesical instillation of Bacille-Calmette-Guerin (BCG) bacteria or cytostatic drugs. While these treatments may result in prolonged tumor free periods, there is a need for less toxic and more specific therapies.

The therapeutic effect of the alpha1-oleate complexes is encouraging, in view of the positive findings in a prior clinical study, where intravesical HAMLET instillations triggered massive tumor cell exfoliation as well as morphological changes in the tumor, including a reduction in tumor size. Toxic effects of HAMLET were not detected and the patients did not report adverse effects. Importantly, the alpha1-peptide-oleate complexes were retained in bladder tissues, suggesting an extended time of contact with the tumor and potentially an extended tumoricidal effect. Furthermore, the retention of active substance was specific for tumor bearing mice and the active substance was detected in tumor tissue. The results identify alpha1-peptide-oleate complexes as bio-similars to HAMLET, with therapeutic activity in the same molar range in the murine MB49 bladder cancer model and selectivity for tumor tissue.

Furthermore, the effect is clearly shared by other peptides derived from membrane perturbing proteins. Proteins that affect membrane curvature are often enriched for alpha-helical structure. Their insertion into spherical lipid membranes may cause tubulation and recruit cytosolic factors to newly created membrane compartments. The applicants have found new role for membrane-integrating, alpha-helical peptides as cancer therapeutics.

The N-terminal alpha-helices of membrane proteins such as Sar1 both gained tumoricidal activity, after forming complexes with oleate in line with that achieved by alpha-lactalbumin alpha peptides. As illustrated below. loss of tumor cell viability was demonstrated in vitro for cancer cells from the lung, kidney and urinary bladder and in vivo in a murine bladder cancer model. Without being bound by theory, it is possible that this “gain-of-function” may aid proteins to diversify their function in different tissue environments and provide tissue-specific solutions to challenges such as “cancer surveillance”.

As the alpha1- and sar1alpha-peptides lack primary sequence homology, the shared effect is defined by their alpha-helical secondary structure and affinity for oleic acid and for cell membranes. Relevance to the intact protein may be debated but the conclusions are supported by extensive structural studies of alpha-lactalbumin, where stable folding intermediates expose alpha-helical domains. In the alpha-lactalbumin molten globule, the polydispersion of conformations has been shown experimentally and modeled computationally.

It is known that the N-terminal fragment of alpha-lactabumin, alpha1 forms a complex with oleate that reproduces the tumoricidal activity of HAMLET. High-resolution NMR and molecular modeling studies now show striking similarity of the peptide-oleate complexes with HAMLET and its family of complexes, suggesting that HAMLET acquires tumoricidal activity by exposure of membrane perturbing, alpha-helical domains. Importantly, the cell death process is unique for the alpha helical peptides and not activated by the native protein, in which these epitopes are not accessible for cellular targets.

The relative shallowness of the predicted energy basins suggests wide conformational heterogeneities that cannot be found in the potential energy basins of native proteins. Hence, common amongst all of these protein-oleate and peptide-oleate complexes is the strikingly malleable nature of the tertiary structures. Without being bound by theory, it appears that this “adapt-and-adopt” fluidity of conformations may be one of the key reasons that the HAMLET and the peptide-oleate complexes described herein are able to act upon a variety of different tumor cell lines and cancer tissues. Such availability of different binding surfaces may allow these peptide-oleate complexes to possess similar properties to intrinsically disordered proteins (or intrinsically unfolded proteins, IDPs), where the binding interactions are found to be generally promiscuous. Unlike conventional IDP's that involve only polypeptide chains, the peptide-oleate complexes display similar properties only with the addition of the lipid cofactor, which, in addition to providing the three-dimensional surface, also endows it with novel function. Due to this post-fatty-acid-binding, gain-of-function property and a lack of pore-formation, these complexes can be distinguished from antimicrobial peptides (AMP) which perturb membranes via mechanisms distinct from known AMPs. Importantly, the cellular effects of the complexes contrast strongly with those of the naked protein/peptides and/or the fatty acid alone, refuting the arguments that HAMLET's tumoricidal activity is due solely to lipid toxicity.

The results suggest that the lack of a fixed, defined structure is crucial for the peptide/protein-oleate complex to interact with tumor cell membranes and enter tumor cells. While the cell death response is initiated at the membrane, it is amplified/propagated inside the tumor cell by multiple specific interactions. Nuclear speckles are important sub-nuclear compartments, which work in concert to coordinate gene expression, including transcription, pre-mRNA processing and mRNA transport. Transcriptionally active genes localize to the speckles, where a continuous and rapid molecular exchange takes place with the surrounding nucleoplasm. We propose that the complexes of the invention including sar1alpha-oleate may, in common with alpha-1-oleate, disturb this stochastic self-organization, by establishing high affinity complexes with histone H3, damaging the architecture of the transcriptional machinery and preventing the dissociation of bound components. These effects were further supported by a direct effect on PKC-dependent phosphorylation of SC35, which in turn, inhibits Pol II activation. The resulting inhibition of gene expression, which involved H3 and proteasome-centric gene networks, may mark a “point of no return” for the dying tumor cell, as de novo synthesis of critical cellular constituents is severely impaired.

In a prior clinical study, intravesical HAMLET instillations triggered massive tumor cell exfoliation as well as a tumor response, seen as a reduction in tumor size. The patients did not report adverse effects and tissue toxicity of HAMLET was not detected, supporting the tumor specificity of HAMLET, also observed in several animal models. Transitional cell carcinomas are common and costly urological malignancies, due to a high recurrence rate and lack of curative therapies. Tumors confined to the mucosa are often treated by transuretheral resection, followed by intravesical instillation of Bacille-Calmette-Guerin (BCG) bacteria or cytostatic drugs. While these treatments may result in prolonged tumor free periods, there is a need for less toxic and more specific therapies. The therapeutic effects of the peptide-oleate complexes are especially encouraging, as they identify sar1alpha- and other alpha peptides as bio-similars to HAMLET, with therapeutic activity in the same molar range and selectivity for tumor tissue.

### DETAILED DESCRIPTION

The invention will now be particularly described by way of example. However, it will be apparent to one skilled in the art that the specific details are not required in order to practice the invention. The following descriptions of specific embodiments of the present invention are presented for purposes of illustration and description. They are not intended to be exhaustive of or to limit the invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. The examples refer to the accompanying diagrammatic drawings in which:

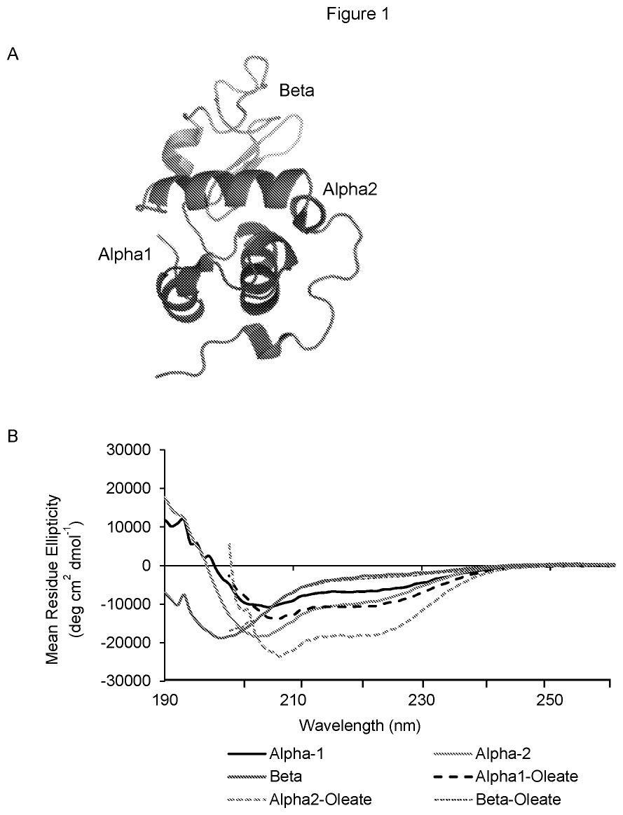

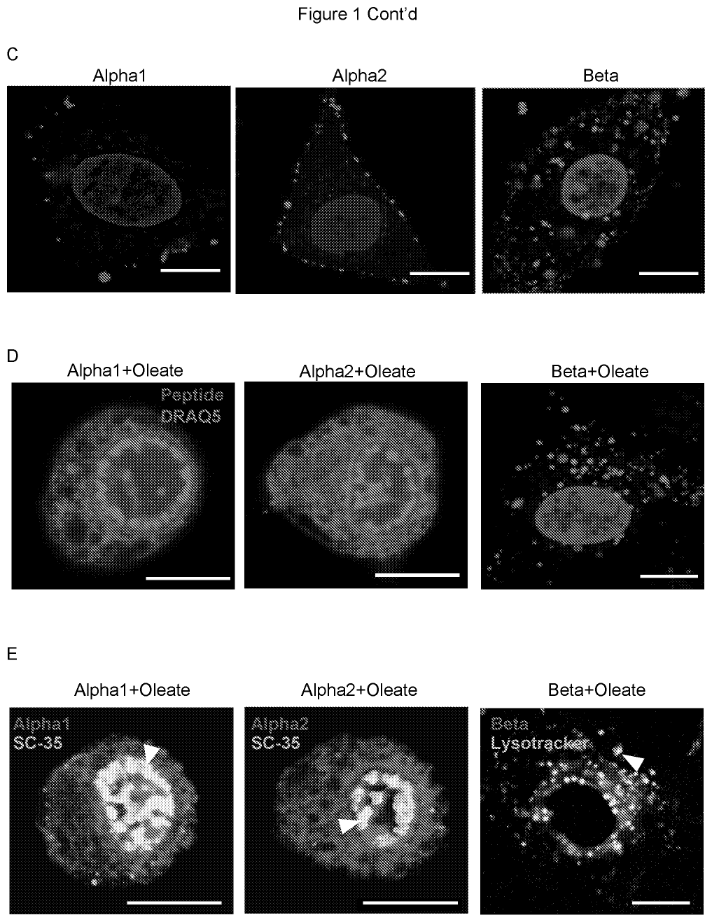

FIG. 1: Peptide-specific interactions with tumor cells.

(a) Crystal structure of human α-lactalbumin, indicating the alpha1, alpha2 and beta sheet domains (PDB id: 1B90). (b) Far-UV circular dichroism analysis identifying alpha-helical secondary structure of the alpha-peptides, which is enhanced in the presence of oleate. (c) Plasma membrane foci of biotinylated alpha1- and alpha2 peptides in lung carcinoma cells (1 hour, 35 uM). The beta-peptide was rapidly internalized into cytoplasmic vesicles. (d) Internalization of alpha1- and alpha2 peptide-oleate complexes by tumor cells and accumulation in a ring-like structure in the nuclear periphery. (e) The alpha1- or alpha2-peptide-oleate complexes co-localized with the nuclear speckles marker, SC-35 in tumor cell nuclei. The beta peptide-oleate complex co-localized with the lysosome marker Lysotracker. Scale Bar, 10 μm.

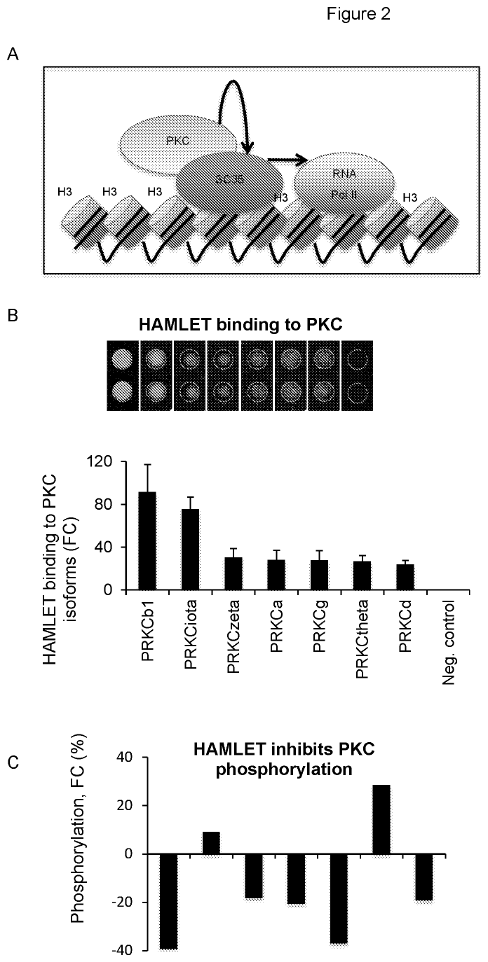

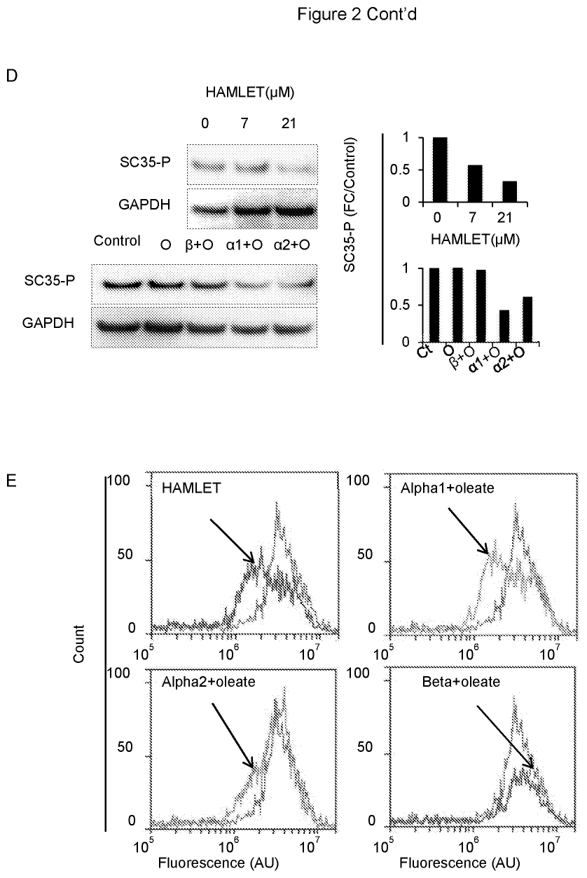

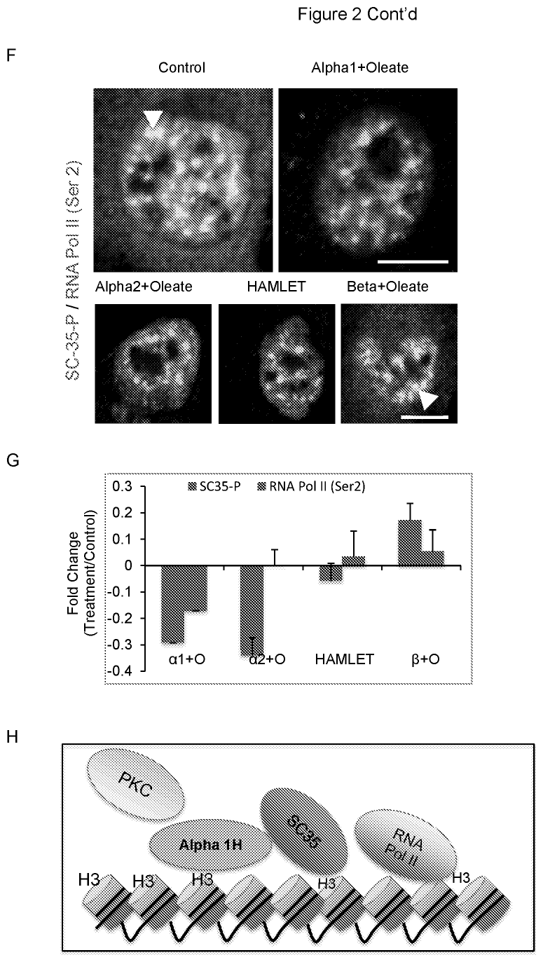

FIG. 2: Molecular interactions in nuclear speckles.

(a) Model from McCuing et al., depicting the interactions of SC-35 with nuclear speckle constituents such as Histone H3, PKC and RNA Pol II (b) Binding of HAMLET to Protein kinase C (PKC) isoforms, detected in a proteomic screen. (c) Inhibition of PKC phosphorylation by HAMLET (5/6 isoforms, 1 hour, 21 uM), defined by a decreased signal in a phospho-antibody array. (d) Inhibition of SC-35 phosphorylation by

HAMLET and the alpha-peptide-oleate complexes. Western blot analysis of whole cell extracts, stained with antibodies to phosphorylated SC-35. GAPDH was used as a loading control. (e) Inhibition of SC-35 phosphorylation quantified by FACS, using specific antibodies. (f) Co-localization of SC-35 and Pol II in cells treated with HAMLET or alpha-peptide-oleate complexes. The loss of co-localization was estimated by the Pearson correlation co-efficient (R). Scale Bar, 5 (g) Quantification of images in (f) supporting a decrease in phosphorylation of SC-35 and RNA Pol II. (h) Model depicting how interactions between nucleosome constituents are perturbed by the HAMLET/alpha-peptide oleate complexes (modified from (a).

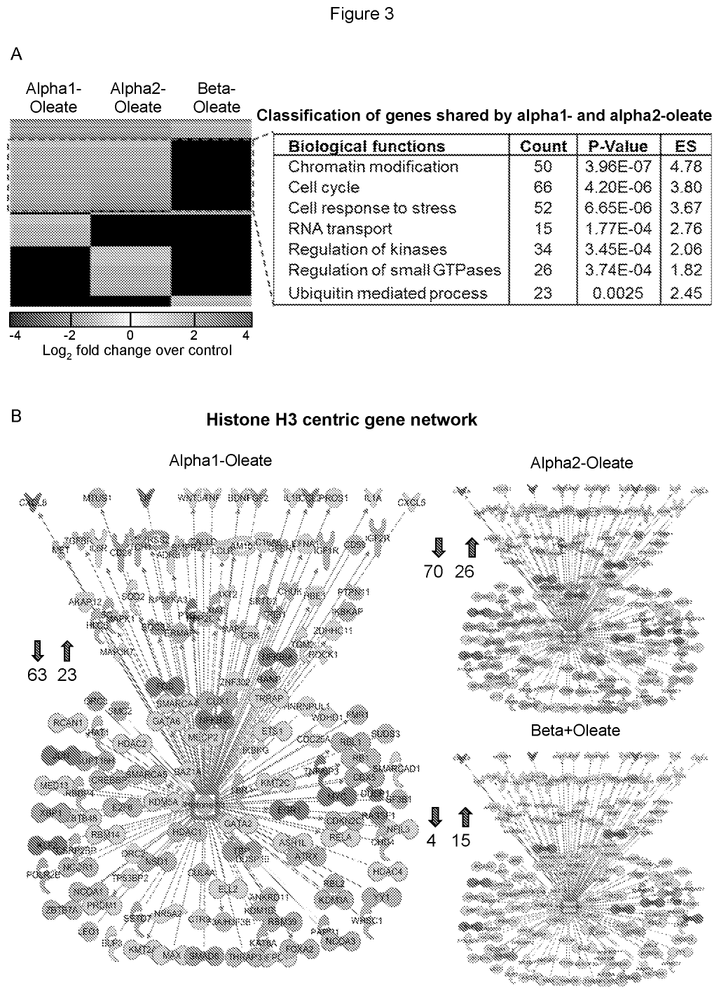

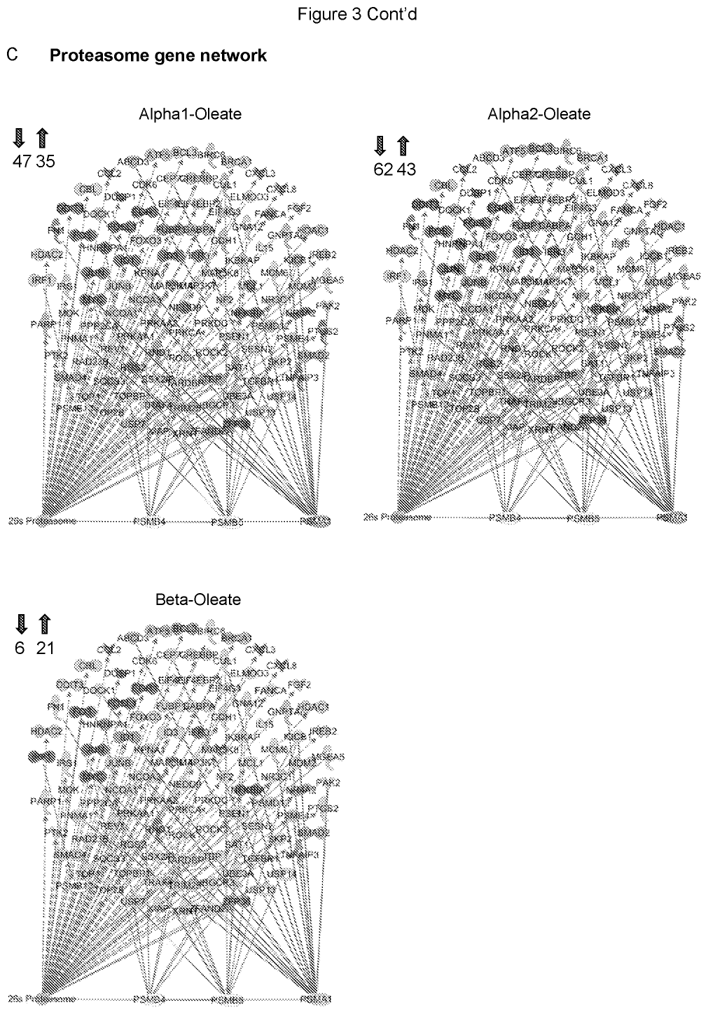

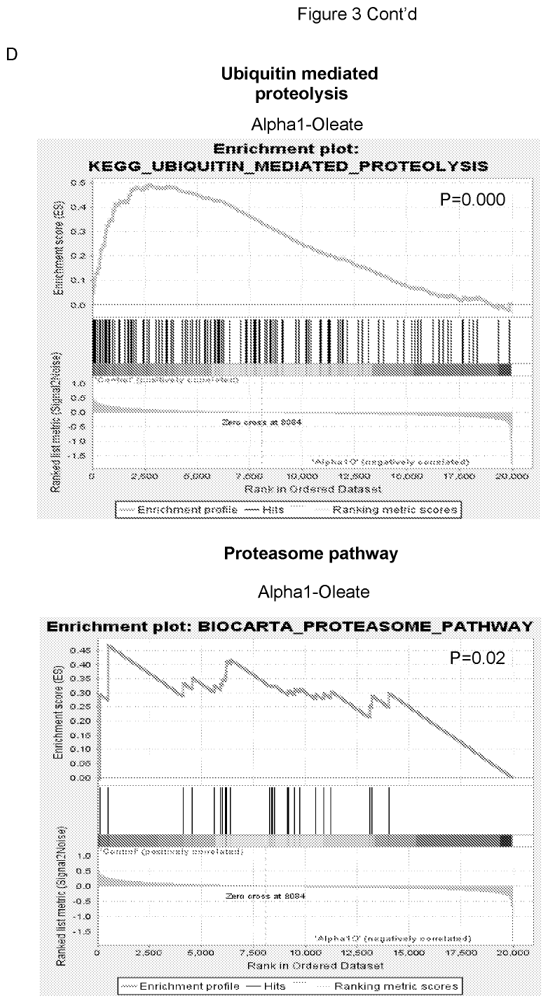

FIG. 3: Inhibition of gene expression: Histone H3- and proteasome-related gene networks. Lung carcinoma cells were exposed to alpha1- or alpha2-oleate complexes and the beta-oleate complex was used as a negative control (35 uM, 1 hour). Extracted RNA was subjected to genome-wide transcriptomic analysis and significantly regulated genes were identified by DAVID. (a) Eight top-scoring biofunctions were co-regulated by the alpha1- and alpha2-oleate complexes and most of the genes were inhibited. Activated genes are shown in dark grey. (b) Histone H3 centric network quantifying the effects of the alpha1- or alpha2-oleate complexes, compared to untreated cells. (c) Proteasome centric network quantifying the effects of the alpha1- or alpha2-oleate complexes, compared to beta-olete complexes. (d) Suppression of genes involved in ubiquitin mediated proteolysis and the proteasome pathway, defined by GSEA. The beta-oleate complex did not significantly affect these end points.

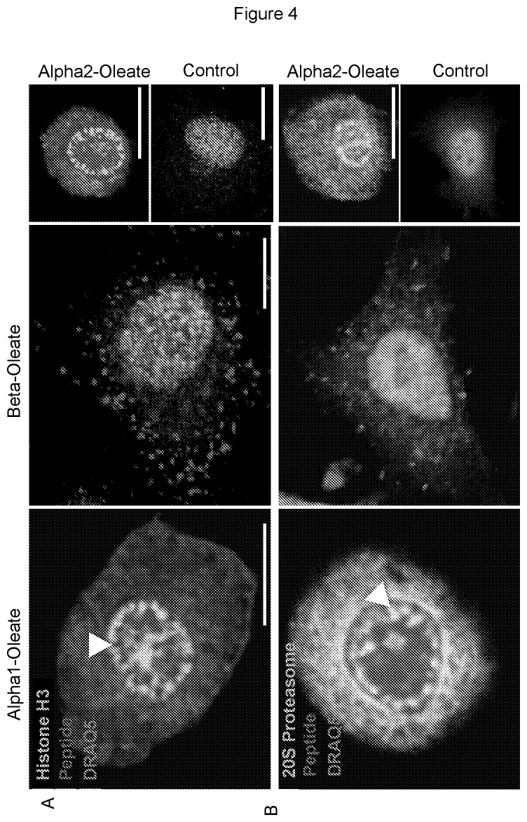

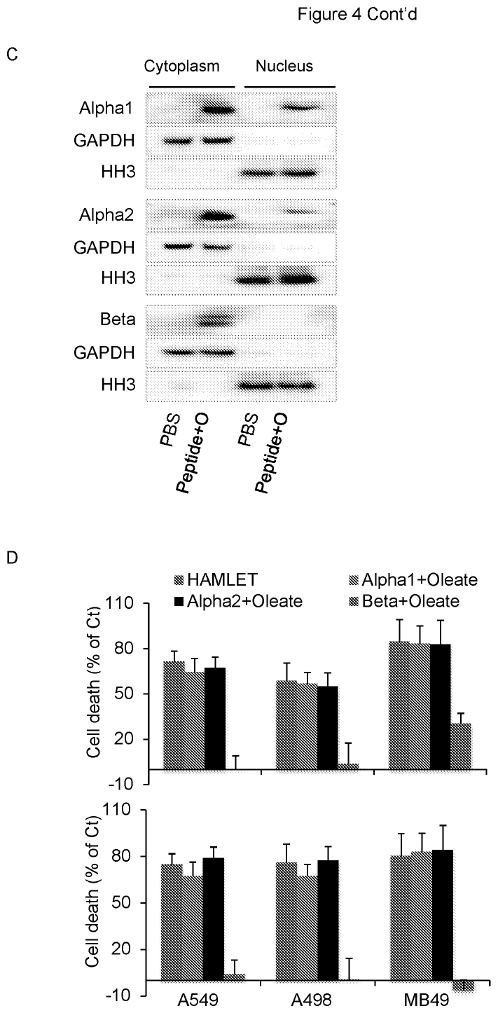

FIG. 4: Constituents of nuclear speckles and tumor cell death. (a) Strong co-localization of the alpha1- and alpha2-peptides with Histone H3 in the nuclear speckles. Counterstaining of DNA with DRAQ-5. (b) Strong co-localization of the alpha1- or alpha2-peptides with 20S proteasomes in nuclear speckles and in the cytoplasm. The beta peptide-oleate complex was restricted to the cytoplasm. Scale Bar, 10 μm. (c) Subcellular quantification of the peptides by Western blots of nuclear and cytoplasmic fractions. Peptides were detected, using peptide-specific antibodies. GAPDH and histone H3 were used as loading controls for the cytoplasmic and nuclear fractions, respectively. (d) Tumor cell death in human lung carcinoma cells (A549), human kidney cells (A498) or murine bladder cancer cells (MB49), treated with alpha1- or alpha2- or beta-oleate complexes (35 uM). HAMLET was used as a positive control. Cell death was quantified as the reduction in total cellular ATP (%) levels or Presto Blue fluorescence (%).

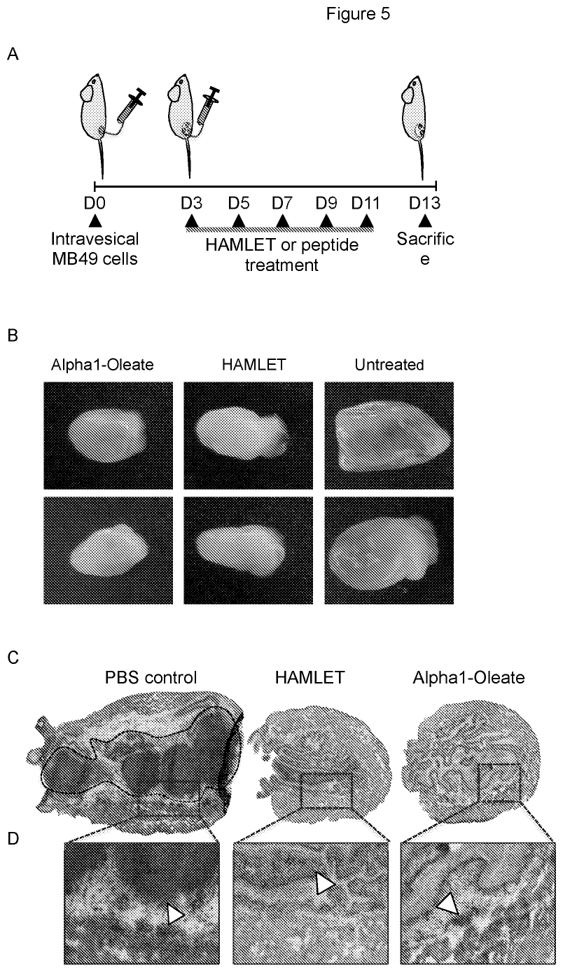

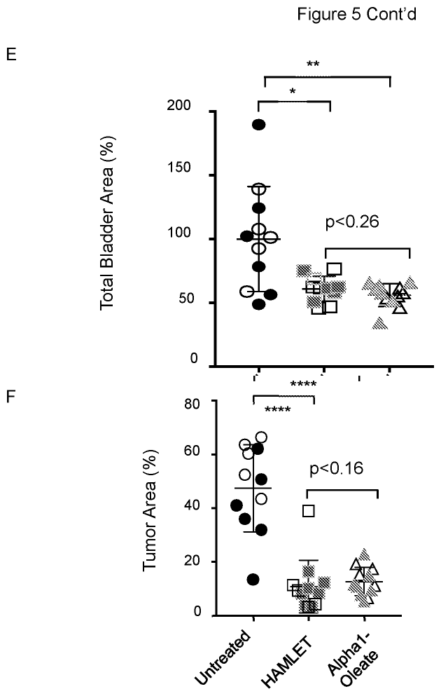

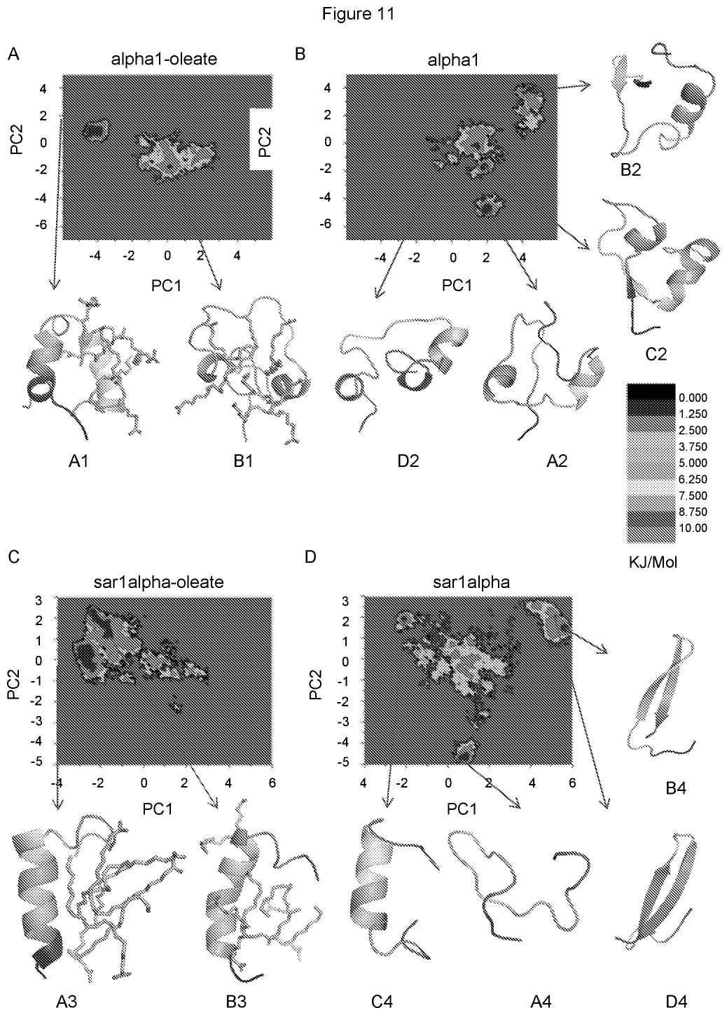

FIG. 5: Therapeutic efficacy of alpha1-peptide oleate complexes in the murine MB49 bladder cancer model. (a) Bladder cancer was established by intravesical inoculation of the rapidly proliferating MB49 cell line⁴⁸. From day 3, mice were treated by repeated intravesical instillations of alpha1-oleate complexes at indicated intervals (n=5, 100 μl each). Tumor development was quantified at sacrifice, on day 13. HAMLET served as a positive and PBS as a negative control. (b) Macroscopic appearance of bladders from mice treated with the alpha1-oleate complex, HAMLET or PBS. (c) Tumors were visualized in whole bladder mounts; stained with H&E. Demarcation against healthy tissue is shown by the dotted line. (d) High magnification images of individual tumor areas (arrow heads) with high cellularity. (e) Therapeutic efficacy quantified as a reduction in bladder size and (d) tumor area in alpha1-oleate and HAMLET treated mice, compared to the PBS control. Experiment 1, n=5, open symbols, Experiment 2, n=6-7, filled symbols. Means±S.D. (f) Real-time in vivo fluorescence imaging shows retention of the VivoTag 680 labeled alpha1 peptide and HAMLET in the lower pelvic region of tumor bearing mice (n=2). Retention of the alpha1-peptide in tumor-bearing mice, indicated by comparison of the signal immediately after inoculation (5 min), compared to 24 hours post instillation. In contrast, the signal was gradually lost in tumor-free control mice (Supplementary FIG. 11). (g) Tumor specific uptake of alpha1-peptide (arrowhead), 24 hours after intravesical inoculation of the Alexa Fluor-568 labeled peptide-oleate complex. Scale bar, 100 μm.

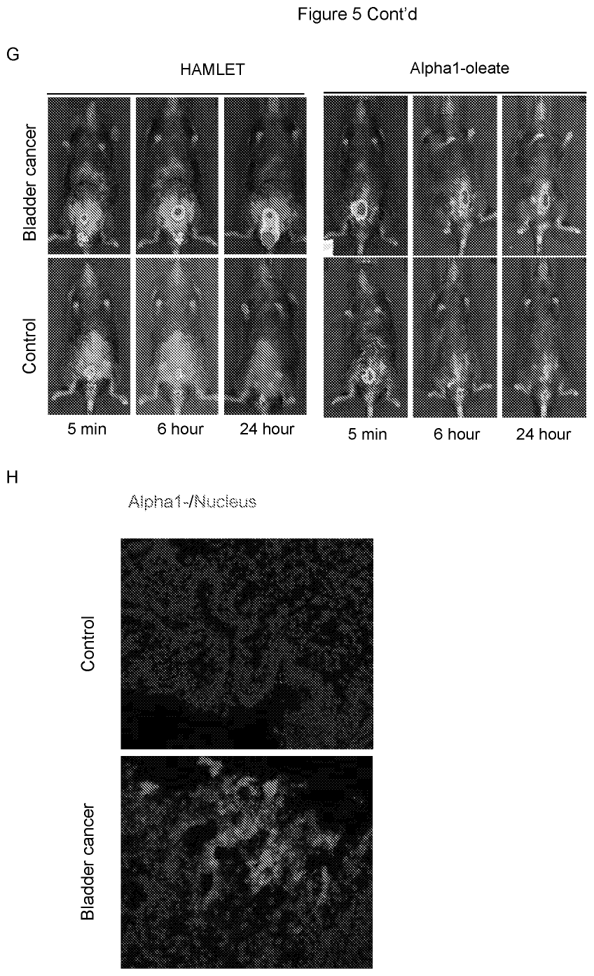

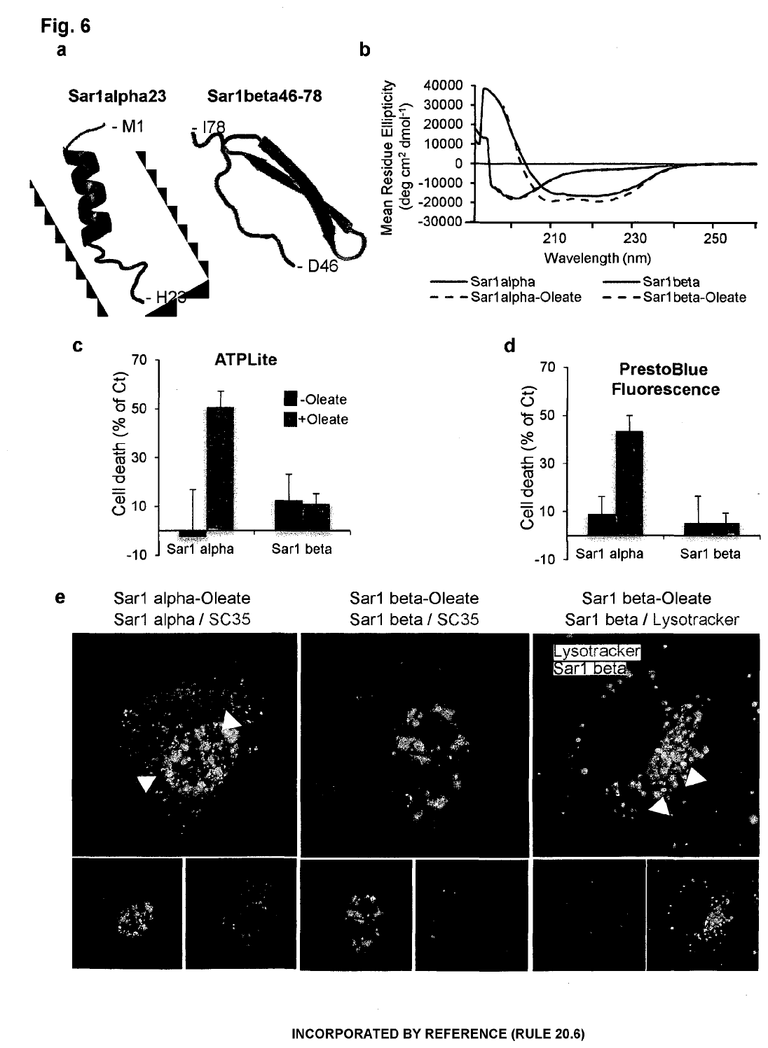

FIG. 6: Tumoricidal activity and nuclear speckle accumulation of the alpha-helical Sar1alpha23-peptide-oleate complex. (a) Structural models for alpha helical and beta peptides. Top I-TASSER models for Sar1-alpha23 and Sar1-beta46-78 peptides, shown in PyMOL's cartoon ribbons representations. (b) Far-UV circular dichroism analysis identified alpha-helical secondary structure of the Sar1alpha1-23 peptide, which is enhanced in the presence of oleate. (c) Tumor cell death in human lung carcinoma cells (A549) treated with Sar1alpha23 or Sar1beta46-78 alone (35 uM) or in complex with oleate (175 uM). Cell death was quantified as the reduction in total cellular ATP levels (%) or Presto Blue fluorescence (%). (d) The Sar1alpha23-oleate complexes co-localized with the nuclear speckles marker, SC-35 in tumor cell nuclei. The Sar1beta46-78 peptide-oleate complex co-localized with the lysosome marker Lysotracker. Scale Bar, 10 μm.

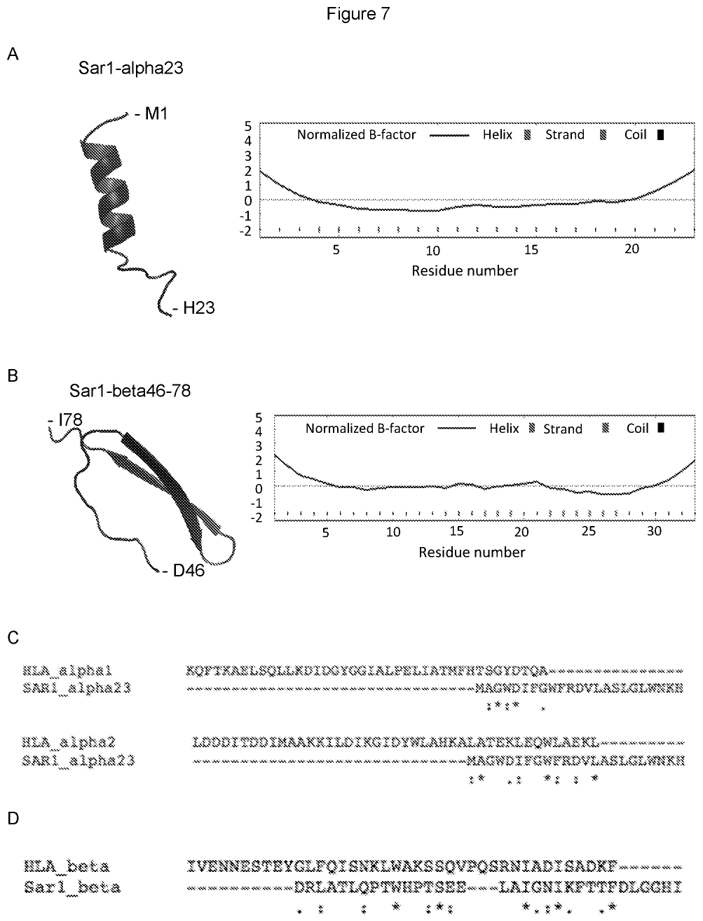

FIG. 7: Structural models of alpha helical and beta domain peptides in Sar1; a COP II family protein. (a-b) Top I-TASSER models for Sar1-alpha23 and Sar1-beta46-78 peptides, shown as PyMOL's cartoon ribbon representations. Left, Residue-specific secondary structure assignment with predicted normalized B-factor (B-factor>1 suggests higher stability) for respective structure. The 3D structures were modeled by I-TASSER, which uses a combination of sequence alignment methods, ab initio modeling and further structure alignment to model proteins, complementing the known crystal structure information. The top I-TASSER models for the peptides were generated with high confidence. For Sar1-alpha23 (TM-score=0.68±0.12 and RMSD=1.8±1.5 Å), about 60% stable helices were predicted (B-factor<1) (Fig. XB). For Sar1-beta46-78, the top model predicted about 30% of beta-sheet fold (TM-score=0.59±0.14 and RMSD=3.6±2.5 Å). (b) Sequence alignment of alpha1 (or alpha2) alpha-lactalbumin peptides or the Sar1-alpha23 Sar1peptide. (b) Sequence alignment for alpha2 peptide from alpha-lactalbumin and Sar1-beta46-78 peptide from Sar1.

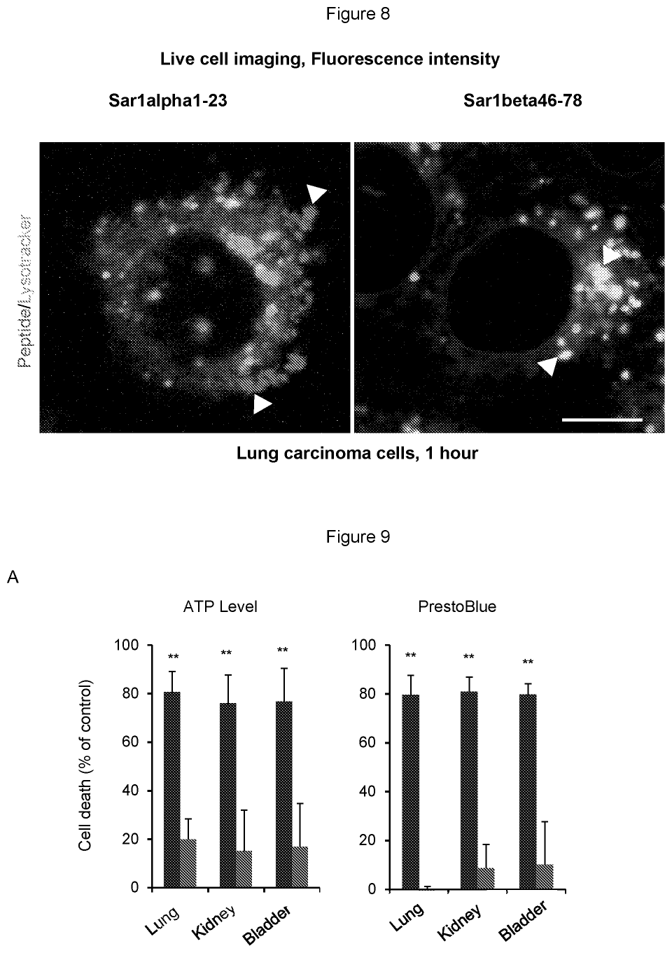

FIG. 8: Lysosomal accumulation of the Sar1beta46-78 peptide. A549 lung carcinoma cells were exposed to the beta sheet peptide of Sar1, using the same concentration as the beta sheet peptide from alpha-lactalbumin. The Sar1beta46-78 peptide was internalized and accumulated in the lysosomes, reproducing the Population II phenotype in HAMLET treated cells. The Sar1alpha23 peptide, in contrast, formed membrane aggregates but no significant co-localization with Lysotracker was observed. Scale Bar, 10 μm.

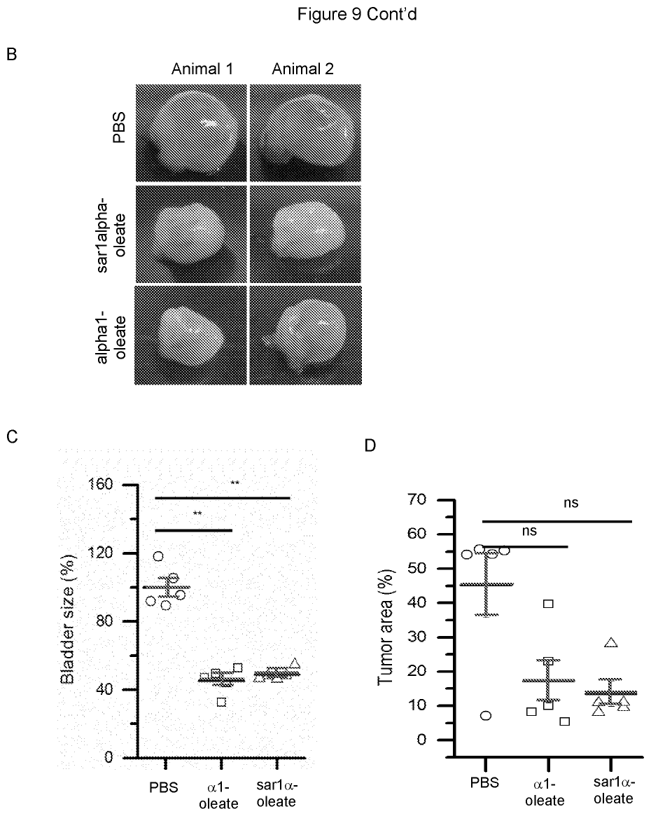

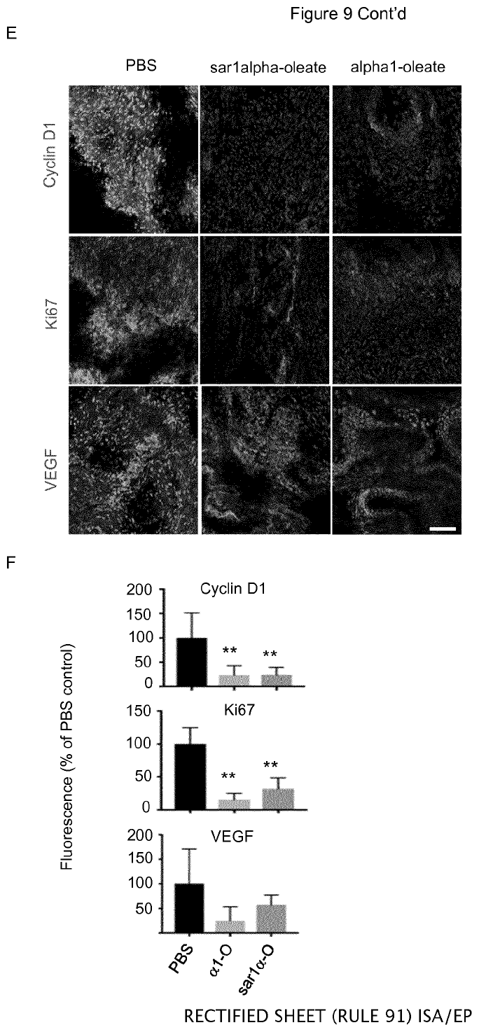

FIG. 9: Sar1alpha Forms Oleate Complexes With Tumoricidal Activity

(A) Lung (A549), kidney (A498) and bladder cancer cells (MB49) were killed by sar1alpha-oleate but not by sar1beta-oleate. Cell death was quantified as the reduction in total ATP levels (% of control) or PrestoBlue fluorescence (% of control). Error bars are means±S.E.M. (n=3, *P<0.05 and **P<0.01).

(B) Macroscopic appearance of bladders from mice treated with sar1alpha-oleate and alpha1-oleate, as a positive control.

(C-D) Therapeutic efficacy of sar1alpha-oleate in the murine MB49 bladder cancer model quantified as a reduction in (C) bladder size and (D) tumor area in sar1alpha-oleate and alpha1-oleate treated mice, compared to the PBS controls. Error bars are means±S.E.M., *P<0.05 and **P<0.01.

(E) Tumor area in whole bladder mounts; stained with H&E.

(F) Reduction of proliferation markers (CyclinD1, Ki67 and VEGF) in tumor bearing mice treated with sar1alpha-oleate or alpha1-oleate. DRAQ5 was used as nuclear marker. Scale Bar, 100 μm.

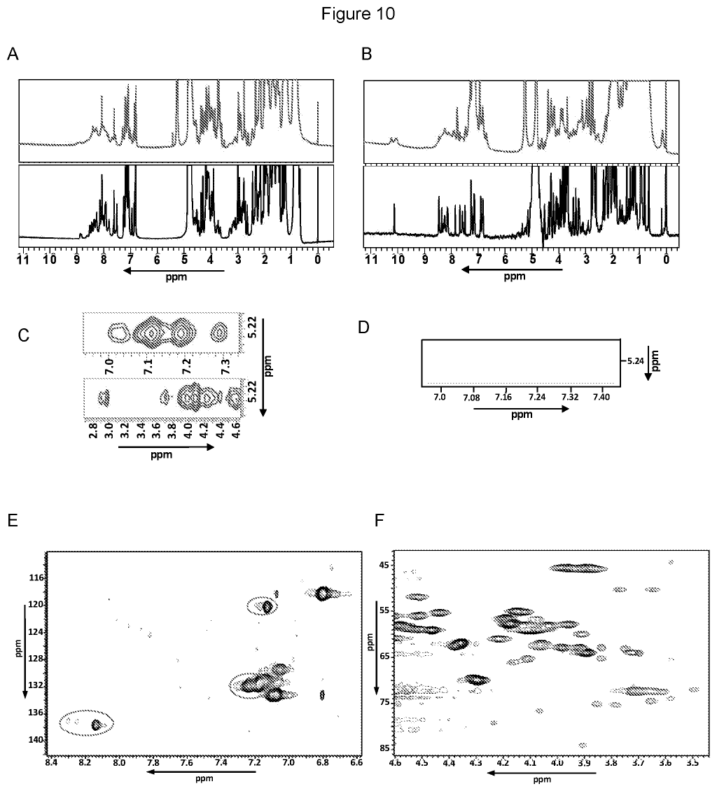

FIG. 10: NMR Spectra Of Alpha1- And Sar1alpha Peptides And Their Respective Complexes

(A) One-dimensional ¹H NMR spectra of alpha1 peptide (black) and the alpha1-oleate complex (grey). The naked alpha1- and sar1alpha-peptides assume a conformationally- and time-averaged ensemble of structures that are interconverting rapidly, and therefore are seen as sharp peaks. (B) One-dimensional ¹H NMR spectra of sar1alpha peptide (black) and the sar1alpha-oleate complex (grey). Note that the peaks are broader in the complexes. The arrows indicate the indole ¹H signals arising from the three Trp side chains present in the sar1alpha peptide. (C-D) Two-dimensional NOESY spectra highlighting nOes between the 9,10 olefinic protons (5.23 ppm) of oleic acid with the Hα protons and aromatic protons of the (C) alpha1-oleate and the (D) sar1alpha-oleate complexes, showing atomic-level proximities of the fatty acid to the respective peptide. (E-F) Overlays of the two-dimensional ¹H-¹³C HSQC spectra of alpha1 (red) and alpha1-oleate (blue) to demonstrate chemical shift perturbation (circled regions) in the (E) aromatic side chain region and the imidazole ring protons, and especially large perturbations in the (F) aliphatic side chain regions, suggesting that a large structural transition has occurred upon binding the fatty acid moieties.

FIG. 11: Free Energy Surface Analyses and representative structures of the Naked Peptide and Peptide-oleate Complexes. (A-D) Free energy surfaces as a function of the first two principal components for (A) alpha1-oleate, (B) naked alpha1, (C) sar1alpha-oleate, and (D) naked sar1alpha. The representative structures of peptides or peptide-oleate complexes, along with their respective local minima annotations).

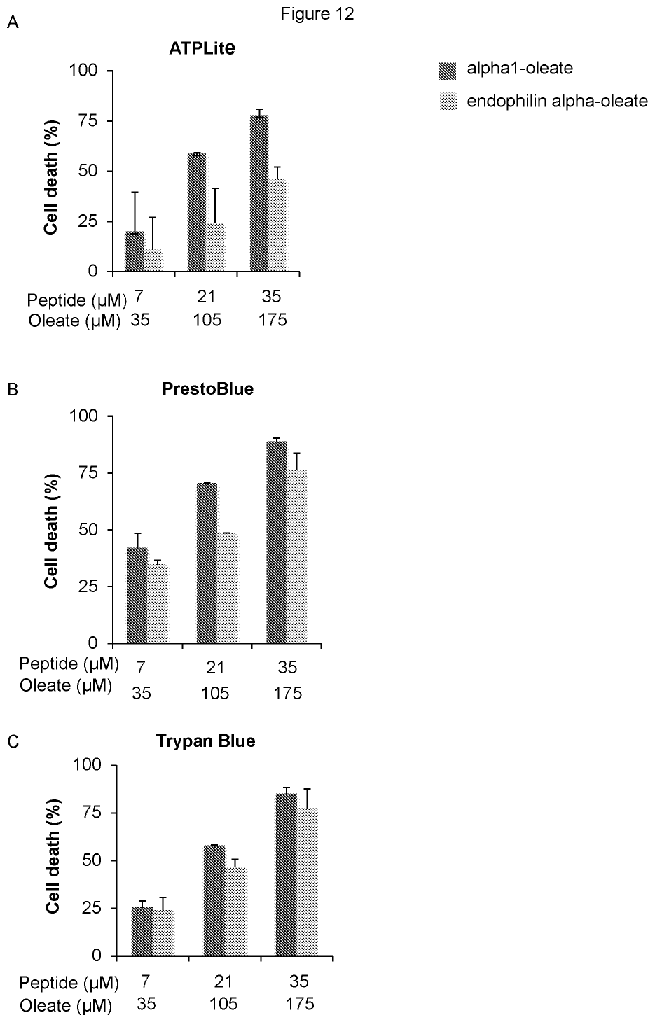

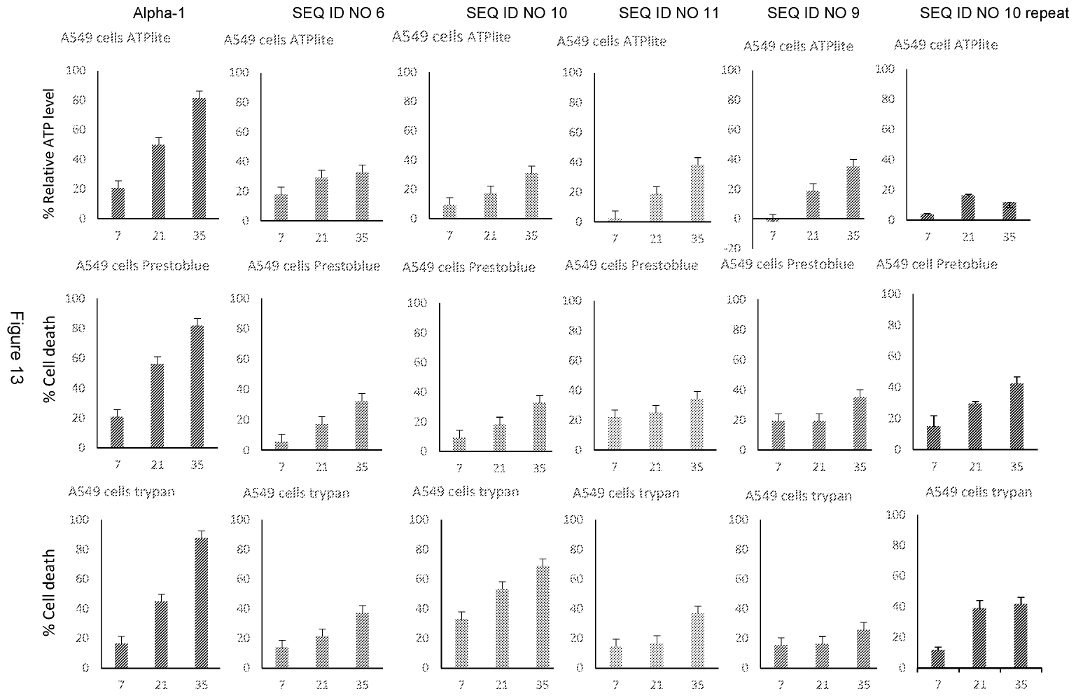

FIG. 12: Shows the results of cell death assays obtained using a complex formed with an endophilin derived peptide (of SEQ ID NO 5), as compared with that obtained using alpha-1-oleate.

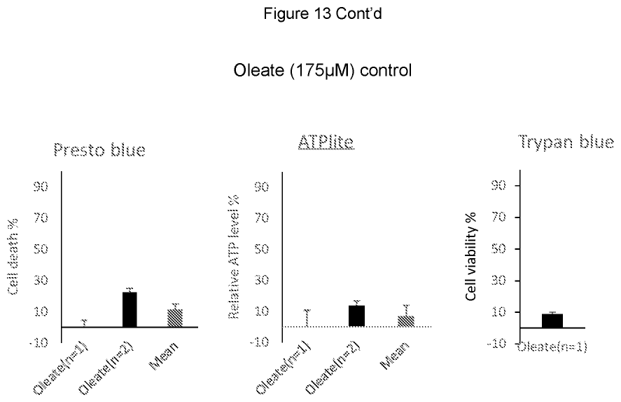

FIG. 13: Shows the results of a cell death assays obtained using a complex formed with a range of peptides derived from membrane perturbing proteins of SEQ ID Nos 6, 9, 10, and 11, as compared with that obtained using alpha-1-oleate and an oleate control.

### Materials and Methods

### Chemicals

DMSO (Dimethyl sulfoxide), Formaldehyde, Triton X-100, Tween-20, Sodium dodecyl sulphate (SDS), Sodium deoxycholate and Fluoromount were from Sigma (St. Louis, Mo.). EDTA (ethylenediaminetetraacetic acid) and Tris (hydroxymethyl) aminomethane were from VWR (Volumetric solutions, BDH Prolabo) and DRAQ-5 was obtained from eBioscience (San Diego, Calif.). RPMI-1640 was from HYclone (HYCLSH30027); sodium pyruvate (11481318), non-essential amino acids (11401378) and fetal bovine serum (10309433) were from Fisher Scientific; gentamicin was from Life Technologies (15710049).

### Peptide Synthesis

The peptides to individual domain of alpha peptides were commercially synthesized using the mild Fmoc chemistry method (Mimotopes, Melbourne, Australia). For biotinylated peptides, an aminohexanoic acid (Ahx) spacer was added to ensure adequate separation between the biotin and the peptide moiety. The sequences for the peptides are as such:

alphaQ:
(SEQ ID NO 1)
Ac-KQFTKAELSQLLKDIDGYGGIALPELIATMFHTSGYDTQ-OH
beta:
(SEQ ID NO 2)
Ac-IVENNESTEYGLFQISNKLWAKSSQVPQSRNIADISADKFLDDD-OH
alpha2:
(SEQ ID NO 3)
Ac-LDDDITDDIMAAKKILDIKGIDYWLAHKALATEKLEQWLAEKL-OH
Sar1alpha23:
(SEQ ID NO 4)
Ac-MAGWDIFGWF RDVLASLGLW NKH-OH
Sar1beta46-78:
(SEQ ID NO 5)
Ac-DRLATLQPTWHPTSEELAIGNIKFTTFDLGGHI-OH

Additional peptides were similarly synthesized and these were:
MSVAGLKKQF HKATQKVSEK VGGAEGTKLD DDFKE (SEQ ID NO 5);
SF SSLGLWASGL ILVLGFLKLI HLLLRRQT (SEQ ID NO 6):
SEKKKTRRANGFKMFLAALSFSYIAKALG (SEQ ID NO 10):
GTPEYVKFAR QLAGGLQALMWVAAAICLIA (SEQ ID NO 11) and
VQIPYEVTLW ILLASLAKIG FHLYHRLPG (SEQ ID NO (9).

### Complex Preparation

5 mg of sodium oleate was dissolved in 1 ml of RPMI to give a 16 mM clear solution, which was mixed with the various peptides in a ratio of oleate:peptide of 5:1.

### Circular Dichroism of Synthetic Peptides

Far-UV CD spectra was collected on alpha1-, alpha2- and beta-peptides with and without oleate at 25° C. using Jasco 815 CD Spectropolarimeter. The peptides were dissolved in 50 mM sodium phosphate buffer, pH 7.4 with 10% D₂O, at a final concentration 0.2 mg/ml. Far-UV CD was performed from 185 to 260 nm for the samples without oleate and from 200 to 260 nm for the samples with oleate. The buffer was subtracted from the values obtained and the mean residue ellipticity (MRE), θm, in deg cm2 dmol-1, was calculated as described previously⁴⁶. The MRE values were then submitted to K2D3, an online server allowing the prediction of secondary structure from far UV CD data⁴⁷. From far-UV circular dichroism (CD) measurement and K2D3 analysis, alpha1- and alpha2-peptides were shown to retain high percentage of alpha helical content, mimicking their native secondary folds. Addition of five molar excess of oleate did not perturb the helical content as spectra corresponding to the alpha peptides showed little changes in 222 nm and 208 nm minima. In comparison, when beta peptide was mixed with oleate, a dramatic shift from random coil to predominantly alpha-helical secondary structure was observed, suggesting that oleate induces a helical transition or folding.

### Cellular Assays

Human lung carcinoma cells (A549, ATCC) and human kidney carcinoma cells (A498, ATCC) or mice bladder carcinoma cells (MB49) were cultured in RPMI-1640 with non-essential amino acids (1:100), 1 mM sodium pyruvate, 50 μg/ml Gentamicin and 5-10% fetal calf serum (FCS) at 37° C., 5% CO₂. For cell death experiment, cells were grown on 96-well plate (2×10⁴/well, Tecan Group Ltd) overnight. Cells were incubated with HAMLET or peptide-oleate complexes in serum-free RPMI-1640 at 37° C. FCS was added after 1 hour. After 3, 7 and 12 hours treatment cell death was quantified by two biochemical methods: Cell viability was quantified by Presto Blue fluorescence (Invitrogen, A13262) and cellular ATP levels using luminescence based ATPlite™ kit (Perkin Elmer). Fluorescence and luminescence was measured using a microplate reader (Infinite F200, Tecan).

### Protein-Protein Interaction Profiling

ProtoArray® Human Protein Microarray version 4.0 (Invitrogen) was performed as previously described²⁰, which consists of approximately 8,000 human proteins. AlexaFluor®568-labeled HAMLET was added to protein arrays at two concentrations (5 and 50 ng/μl) in duplicate, and the fluorescence was measured by using GenePix 6.0. Negative control array was incubated with buffer alone and scanned at a wavelength of 532 nm. Positve control array was incubated with V5-tagged yeast calmodulin kinase 1, known to exhibit a specific interaction with calmodulin, which is printed in every subarray, was subsequently incubated with the detection reagent AlexaFluor®647-labeled anti-V5 antibody and scanned at a wavelength of 635 nm. The interactions were quantified as fold change (FCs) over the average negative control value.

### In Vitro Kinase Activity Assay

The in vivo kinase activity assay was performed using Kinex™ KAM-850 Antibody Microarray Services (Kinexus, Canada). Untreated and HAMLET-treated samples were performed in duplicates. Raw quantification data and basic analyses for individual samples were provided. Reference data, consolidated data of over 200 samples was also provided as reference, confirming the validity of the assay performed. Targets with percent fold change over control>10 were significant. The targets were identified in the human profiling human activity-based phosphorylation network (Molecular Systems Biology 9:655).

### Live Transmission Light Microscopy Imaging

Lung carcinoma cells detached with Versene were suspended in serum free RPMI medium (5×10⁵ cells/ml). The cells were seeded on a cover slip and allowed for partial adherence for 10 min at room temperature prior to peptide-oleate complexes treatment. Immediate changes in cell morphology were captured using LSM 510 META confocal microscope (Carl Zeiss) using 40× oil immersion objective. The 633 nm HeNe laser and a 650 long-pass filter were used for the image acquisition.

### Confocal Imaging

Cells were grown on 8-well chamber slide (3×10⁴/well, Lab-Tek) overnight. Lung carcinoma cells were treated with HAMLET or peptides (35 μM, 10% Alexa Fluor-488 or −568 labeled). Labeling was done via amine coupling according to manufacturer's instructions (Life Technologies). After treatment, cells were fixed with 2% paraformaldehyde, permeabilized with Triton X-100 (0.25% in PBS) for 10 min, washed with PBS and blocked with 10% FCS in PBS for 10 min at room temperature. Cells were then incubated with anti-Histone H3 (ab1791, Abcam), anti-SC-35 (ab11826, Abcam), anti-Ras (ab108602, Abcam) or anti-20S proteasome (PW8155, Enzo Life Sciences) antibodies (1:50 in 10% FCS/PBS) for 2 h at room temperature, washed three times with PBS and incubated with appropriate secondary antibodies conjugated to Alexa-488 (1:100 in 10% FCS/PBS, Molecular Probes) for 1 hour at room temperature. Nucleus was stained with DRAQ-5 (ab108410, Abcam). Cells were washed with PBS three times and mounted using Fluoromount. Slides were examined using LSM 510 META laser scanning confocal microscope (Carl Zeiss).

### Cellular Uptake of HAMLET or Peptides

For uptake experiments lung carcinoma cells were treated with Alexa-488 or 568 labeled HAMLET, washed and visualized under confocal microscope live. Localization of HAMLET in lysosomes was detected by pre-labelling the cells with lysotracker (LysoTracker Green DND-26, Thermo Fisher). For peptide or peptide-oleate complexes uptake, lung carcinoma cells were treated with individual biotinylated peptide alone or mixed with sodium oleate. Cells were fixed (2% PFA, 10 min), permeabilized (0.25% Triton X-100) and blocked with FCS (10% in PBS). The biotinylated peptides were detected with Alexa-488 or 568 labeled streptavidin conjugate (1:200, 5% FCS/PBS, 1 hour). The accumulation of peptide or peptide oleate complexes in lysosomes was investigated by treatment of lung carcinoma cells with Alexa-568 labeled peptide (20%) or peptide-oleate complexes for 1 hour. Cells were counter stained with lysosomal marker, Lysotracker. Slides were examined using LSM 510 META or LSM 800 laser scanning confocal microscope (Carl Zeiss).

### Western Blotting

Cells were grown on 6-well plates (3×10⁵/well, TPP) overnight. Cells treated with HAMLET were lysed with mammalian NP-40 lysis buffer supplemented with protease and phosphatase inhibitors (both from Roche Diagnostics). HAMLET was detected using goat anti-bovine α-lactalbumin antibody (A10-128P, Bethyl) and synthetic peptides corresponding to the respective alpha and beta domains of partially unfolded α-lactalbumin were detected using peptide specific antibodies (GeneCust). The anti-GAPDH (1:1,000, sc-25778, Santa Cruz) and anti-Histone H3 (ab1791, Abcam) were used as loading controls. Primary antibodies followed by HRP-conjugated secondary anti-rabbit or anti-mouse antibodies (1:4000, 5% NFDM, Cell Signaling) and visualized using ECL Plus detection reagent (GE Health Care). Band intensities were quantified using the ImageJ software 1.46r (NIH).

### Transcriptomic Analysis

Lung carcinoma cells (300,000/well) were allowed to adhere overnight on a 6-well plate (TPP, Trasadingen, Switzerland). After treatment with individual peptide-(35 μM) oleate (175 μM) complexes for one hour, total RNA was extracted (RNeasy Mini kit, Qiagen). 100 ng of RNA was amplified using GeneChip 3'IVT Express Kit and fragmented and labeled aRNA was hybridized onto Human Genome (HG)-219 array strips for 16 hours at 45° C., washed, stained and scanned using the Geneatlas system (all Affymetrix). Transcriptomic data was normalized using Robust Multi Average implemented in the Partek Express Software (Partek). Fold change was calculated by comparing treated cells to PBS control cells. Genes with absolute fold change>1.41 were considered differentially expressed. Heat-maps were constructed by Gitools 2.1.1 software. Differentially expressed genes and regulated pathways were analyzed using Ingenuity Pathway Analysis software (IPA, Qiagen), String-db and Gene Set Enrichment Analysis (GSEA, Broad Institute).

### Bladder Cancer Model

C57BL/6 female mice were bred at the department of laboratory medicine and used at ages 7 to 12 weeks. For intravesical instillation of MB49 cells and treatments, mice were anesthetized by intraperitoneal injection of ketamine and xylazine cocktail. MB49 tumors were established as described previously⁴⁸. Briefly, on day 0 the bladder was emptied and preconditioned by intravesical instillation of 100 μl poly-L-lysine solution (0.1 mg/ml) through a soft polyethylene catheter (Clay Adams, Parsippany, New Jersey) with an outer diameter of 0.61 mm for 30 minutes before MB49 tumor cells (1×10⁵ in 100 μl PBS) were instilled. Five HAMLET (1.7 mM), peptides (0.85 mM) or PBS instillations, 100 μl each were done at day 3, 5, 7, 9 and 11. Mice remained under anesthesia on preheated blocks with the catheter in place to prolong tumor exposure to HAMLET or peptides (approximately 1 hour). Groups of 5 mice for each treatment and control were sacrificed at each time point, and bladders were imaged and processed for histology.

### Histology

Bladders were embedded in O.C.T. compound (VWR) and 5-μm-thick fresh cryosections on positively charged microscope slides (Superfrost/Plus; Thermo Scientific) were fixed with 4% paraformaldehyde or acetone-methanol (1:1 v/v). For hemotoxylin-eosin (H&E) staining Richard-Allan Scientific Signature Series Hematoxylin 7211 and Eosin-Y 7111 (Thermo Scientific) were used to counterstain the tissue sections. Imaging was done with AX10 (Zeiss).

### Real-Time In Vivo Fluorescence Imaging of HAMLET or Peptides

HAMLET or peptides were labeled using VivoTag 680XL Protein Labeling Kit (Perkin Elmer). Mice were anaesthetized using Isofluorane and 100 ul solution of labeled HAMLET or peptide was instilled in bladders of tumor bearing or healthy control mice.

Hair was removed from the ventral sides of anesthetized mice. Mice were imaged at various time points using an IVIS Spectrum imaging system (Perkin Elmer). Signals from HAMLET or peptides were acquired at fluorescent settings with 680 nm excitation. For tissue specific uptake, Alexa-568 labeled peptide was instilled in bladders of tumor bearing or healthy control mice. Mice were sacrificed after 24 hours of treatment and bladder sections were imaged using Zeiss AX10 fluorescence microscope.

### Statistical Analysis

Results are presented as a Mean±SD. Statistical analysis was performed using Student's t-test or Mann-Whitney test at different statistical levels of significance, *P<0.05 and **P<0.01. Pearson product-moment correlation coefficient, R, was performed for co-localization analysis.

### Illustrative Example 1

### HAMLET is Internalized Into Two Distinct Populations of Tumor Cells

During live cell imaging of Alexa-Fluor labeled HAMLET (35 μM, 1 hour) and adherent lung carcinoma cells (A549), two distinct cellular staining patterns (FIG. 1a) were detected. Population I (51%) showed membrane blebbing, diffuse cytoplasmic staining and accumulation of HAMLET in nuclear speckles, defined by staining with antibodies to the nuclear speckle marker SC-35. Nuclear speckles reside in the inter-chromatin space of eukaryotic nuclei and serve as important nodes in the splicing of pre-mRNA and transport of spliced RNA. In Population II (47%), staining was exclusively cytoplasmic with uptake into vesicles defined as lysosomes. By Western blot analysis of whole-cell extracts, the uptake of HAMLET was shown to be time- and dose dependent.

### Illustrative Example 2

### Peptides Determine the Cellular Distribution of HAMLET

To examine if specific protein domains explain these patterns, synthetic peptides corresponding to the alpha1 domain (residues 1-39) (SEQ ID NO 1), the beta sheet (40-80) (SEQ ID NO 2) or the alpha2 domain (SEQ ID NO 3) (residues 81-123) of alpha-lactalbumin, but lacking in cysteine residues; the globular, 14.2kDa protein constituent of HAMLET (FIG. 1a) were used. Synthetic alpha1- and alpha2-peptides retained a high percentage of alpha helical secondary structure with or without bound oleate. The beta-peptide gained alpha-helical properties when in complex with oleate (FIG. 1b).

As oleate complexes, the alpha1- and alpha2 peptides reproduced the Population I phenotype, with membrane blebbing, diffuse cytoplasmic staining and accumulation in nuclear speckles (FIG. 1c-d). The initial membrane integration phase was peptide-specific but the subsequent internalization and nuclear accumulation of the alpha1- and alpha2 peptides required sodium oleate. The beta peptide, in contrast, reproduced the Population II phenotype of HAMLET-treated cells and accumulated in the lysosomes (FIG. 1e). Oleate was not required for lysosomal accumulation of the beta-peptide and even as oleate complexes, the beta-peptide did not reach the nuclei or affect speckle formation.

These mutually exclusive phenotypes strongly support the hypothesis of peptide domain specific tumor cell recognition.

### Illustrative Example 3

### Effects of Alpha-Helical Peptide-Oleate Complexes on Nuclear Speckle Constituents

Peptide-specific targets were subsequently identified in the nuclear speckles of Population I. Based on imaging data, McCuaig et al Frontiers in immunology 6, 562, doi:10.3389/fimmu.2015.00562 (2015) have constructed a hypothetical model, which predicts that PKC phosphorylates SC-3 5, which then activates the phosphorylation of RNA polymerase II (RNA Pol II) at Serine 2 (Ser2), (FIG. 2a). The applicants therefore hypothesized that HAMLET and the alpha-peptides modify the speckle environment by direct interactions with speckle constituents, which are critical for transcriptional activity, including protein kinase C (PKC) and RNA Polymerase II (Pol II).

In a proteomic screen of 8000 human proteins reported previously (Satoh, J. et al., Journal of neuroscience methods 152, 278-288, doi:10.1016/j.jneumeth.2005.09.015 (2006), the known affinity of HAMLET for histone H3 was confirmed and several PKC isoforms identified as HAMLET targets (FIG. 2b). A decrease in PKC phosphorylation was subsequently detected, using an antibody array specific for phosphorylated kinases and substrates (FIG. 2c). In addition, SC-35 phosphorylation was inhibited by the alpha peptide-oleate complexes and by HAMLET, as shown by Western blot analysis and flow cytometry (FIG. 2d-e). The alpha peptide-oleate complexes also reduced the interaction between the activated SC-35 and Pol II, as shown by confocal imaging, after staining with SC-35 and Pol II Ser 2-specific antibodies (FIG. 2f-g).

The results suggest that HAMLET inactivates SC-35 by an alpha-helical domain specific mechanism involving binding to transcriptionally active chromatin via histone H3 and interference with PKC-dependent phosphorylation of SC-35, resulting inhibition of Pol II phosphorylation (FIG. 2h).

### Illustrative Example 4

### Transcriptional Regulation by the Alpha Peptides-Oleate Complexes

As transcriptionally active genes reside in nuclear speckles, the effects on gene expression was quantified. A total of ˜1350 genes were suppressed by the alpha1- or alpha2-oleate complexes compared to 230 genes in cells treated with the beta-oleate complex. Interestingly, biological function analysis identified chromatin remodeling as a top regulated gene cluster, including pronounced inhibition of histone-related functions (FIG. 3a). A histone H3 centric network was significantly enriched for genes involved in chromatin modification, histone modification and nucleosome organization (FIG. 3b). The transcriptomic analysis of H3-dependent gene networks also revealed a pronounced inhibitory effect on Pol II dependent gene expression, supporting the effects of SC-35 inhibition. In addition, a proteasome-centric network was affected and the suppression of proteasome activation and ubiquitin-mediated proteolysis was confirmed by gene set enrichment analysis (FIGS. 3c and d). Genes involved in RNA transport, cellular stress, Ras and kinase signaling were also mainly inhibited (FIG. 3a) as previously reported.

The results identify the alpha-domain oleate complexes as potent regulators of gene expression in cancer cells. In addition, nuclear uptake of the alpha1- and alpha2 peptides was accompanied by an increase in speckle size and SC-35 staining changed from a more diffuse nuclear pattern to the distinct, ring-like structure. It appears therefore that HAMLET recruits other molecules to the speckles, in particular molecules that bind strongly to the alpha-peptides. The alpha1- and alpha2-oleate complexes triggered a redistribution of H3 to the nuclear speckles, where strong co-localization with histone H3 was detected. In addition, a rapid redistribution of cytoplasmic and nuclear 20S proteasomes to the nuclear speckles was detected and the peptides were shown to co-localize with the 20S proteasomes, in these structures (FIG. 4a-b). Nuclear speckles have previously been shown to contain histones, which are exposed in transcriptionally active chromatin and 20S proteasome recruitment is important for proteasome function. Nuclear and cytoplasmic accumulation of the alpha-peptides was confirmed by western blot analysis. In contrast, the beta-peptide was enriched in the cytoplasmic fraction (FIG. 4c).

### Illustrative Example 5

### Peptide-Specific Death in Tumor Cells of Different Tissue Origins

In preparation for in vivo studies, the tumoricidal effect of the peptide-oleate complexes on tumor cells from different tissues was examined. In addition to the A549 lung carcinoma cells, human kidney cells (A498) were used and murine bladder cancer cells (MB49). Each cell line was treated with HAMLET or an alpha1-, alpha2- or beta-oleate mixtures and the loss of viability was quantified as a decrease in cellular ATP levels or PrestoBlue fluorescence (FIG. 4d).

The alpha1- and alpha2-oleate complexes triggered a rapid reduction in Prestoblue fluorescence in the three cell types with kinetics similar to HAMLET (35 μM). ATP levels varied, with the most rapid response in MB49 cells. The beta peptide-oleate complex had a weaker effect on tumor cell death (FIG. 4d).

### Illustrative Example 6

### Therapeutic Effect of Peptide-Oleate Complexes in a Bladder Cancer Model

Based on the sensitivity of the MB49 urothelial carcinoma cells, the murine bladder cancer model described above was used for studies of therapeutic efficacy. Bladder cancer was induced in C57BL/6 mice, by instillation of MB49 cells on day 0, after preconditioning of the bladders with poly-L lysine for 20 minutes. The mice received five intravesical instillations of the alpha1-peptide-oleate complexes or PBS on days 3, 5, 7, 9 and 11 and on day 13, bladders were harvested for macroscopic evaluation and tissue imaging (FIG. 5a-b). The tumor size was determined by H&E staining of whole bladder mounts (FIG. 5c-f). HAMLET was included as a positive control and sham-treated mice received PBS (two experiments, 5+7 mice per group).

The mice developed palpable tumors and the macroscopic appearance of the bladders was altered in tumor bearing mice, compared to controls that had not received MB49 cells. The tumors were growing from the bladder lumen into mucosal and submucosal tissues, and the tumor mass gradually filled the bladder lumen and replaced functional bladder tissue. The tumors showed increased nuclear density and a loss of tissue structure definition, including mucosal folds (H&E staining, FIG. 5d).

This phenotype was markedly attenuated after treatment with the alpha1 peptide-oleate complexes, with a significant reduction in tumor size from about 60% to <20% (P<0.01, compared to untreated mice). In addition, bladder tissue organization was retained in treated mice. The therapeutic efficacy of the alpha1-oleate complex was comparable to the effect of HAMLET (n.s., FIG. 5e).

### Illustrative Example 7

### Tumor Specific Uptake of the Alpha-Peptide-Oleate Complexes

The retention of alpha-peptide oleate complexes by the tumor was visualized, by in vivo imaging in mice with palpable tumors. VivoTag 680XL labeled alpha1 peptide-oleate complexes were instilled intra-vesically on day 8 after the inoculation of MB49 cells and the fluorescence signal was monitored longitudinally, using the IVIS spectrum in vivo imaging system. The fluorescence signal of the complex was clearly visible in the lower pelvic area and the signal was more pronounced in tumor-bearing mice than in controls without MB49 cells (FIG. 5g). The peptide was retained in tumor-bearing mice, where the signal remained elevated after 24 hours (FIG. 5g). Bladders from control mice showed a rapid loss of the labeled complex, resulting in a weak signal after 24 hours.

To verify that the alpha-peptides are retained by the bladder tumor, Alexa Fluor-568 labeled alpha1 peptide-oleate complexes were instilled into the bladders of tumor bearing mice (day 8, palpable tumors). Significant, tumor-specific accumulation of the alpha-peptide was detected in frozen tissue sections, obtained after 24 hours (FIG. 5h). Furthermore, differences in peptide uptake between the tumor and healthy tissue were detected in tissue areas that contained both tumor and adjacent healthy tissue.

The results identify the alpha1 peptide-oleate complex as a new therapeutic agent in bladder cancer, with significant, tumor specific effects.

### Example 8

### α-Helical, Membrane-Interacting Peptides Become Tumoricidal When Mixed With Oleate

The results above suggest that alpha-lactalbumin acquires tumoricidal activity, by exposure of membrane perturbing alpha-helical domains and binding of oleate. To address if this “gain-of-function” might be a general feature of membrane interacting alpha-helical peptides, peptides based upon Sar1, which is a member of the COPII complex was also investigated. Like HAMLET, Sar1 alters membrane curvature and induces tubulation by membrane insertion of the N-terminal amphipathic a-helix. The N-terminal alpha helical peptide Sar1-alpha23 was therefore synthesized (SEQ ID NO 4) and compared to Sar1-beta46-78 peptide (SEQ ID NO 5), which forms a beta-sheet in the native structure. Structural prediction and circular dichroism spectroscopy measurements showed that Sar1-a was predominantly alpha-helical with or without bound oleate. Sar1-β was partially helical and slightly more helical with bound to the fatty acid (FIG. 6a-b and FIG. 7).

The Sar1-alpha23 peptide gained tumoricidal activity when mixed with oleate, at the 1:5 molar ratio, previously defined for the alpha-lactalbumin peptides. ATP concentrations and Prestoblue fluorescence was reduced by about 50%, after 3 hours, suggesting similar kinetics and efficiency as the alpha-lactalbumin peptides. Diffuse cytoplasmic uptake of the Sar1-alpha23-oleate complex was accompanied by accumulation in nuclear speckles, defined by co-localization with SC-35 (FIG. 6e). The Sar1 beta46-78 peptide, in contrast, reproduced the vesicular cytoplasmic staining pattern of Population II, defined by its co-localization with the Lysotracker (FIG. 6d and FIG. 8).

The sar1alpha-oleate complex was shown to reproduce the tumoricidal effects of alpha1-oleate in the various tissue types using the methodology described in illustrative Example 5 above. ATP concentrations and PrestoBlue fluorescence were reduced by sar1alpha-oleate, to about 20%, after 3 hours, suggesting similar kinetics and efficiency as the alpha1-oleate (FIG. 9A). Sar1alpha-oleate also triggered rapid remodeling of tumor cell membranes and accumulated in nuclear speckles, where SC35 phosphorylation was inhibited, as shown by Western blot analysis and flow cytometry. Furthermore, sar1alpha-oleate reduced the interaction between activated SC35 and RNA Pol II, as shown by confocal imaging.

The results demonstrate that membrane-perturbing, alpha helical peptide-oleate complexes share the ability to invade and kill tumor cells of diverse origin, and in particular that the sar1alpha-oleate complex activates a mechanism of tumor cell death similar to alpha1-oleate. In contrast, the sar1beta-oleate complex did not trigger any of these effects (FIG. 6B).

### Example 9

### Therapeutic Effect of sar1 Alphapeptide-Oleate Complex in a Murine Bladder Cancer Model

Therapeutic activity of the sar1alpha-oleate complex was demonstrated in the murine bladder cancer model, described in illustrative Example 6 above and FIG. 5A. The evaluation was investigator blinded, across biological replicates. Tumor growth was attenuated in mice treated with sar1alpha-oleate and bladder tissue organization was more intact than in sham-treated mice (P<0.01, FIGS. 9B-9F). The therapeutic efficacy was comparable to alpha1-oleate (n.s., FIGS. 9C-9D). Mice treated with sar1alpha-oleate or alpha1-oleate complexes also showed significantly reduced expression of tumor proliferation markers Cyclin D1, Ki-67 and VEGF, quantified by immunohistochemistry of frozen tissue sections (FIGS. 9E, 9F).

The results identify sar1alpha-oleate as a second alpha-helical peptide-lipid complex with tumor specificity and therapeutic potential. The results suggest that certain membrane-interacting alpha-helical peptides may share the ability to form tumoricidal complexes with oleate.

### Example 10

### Biomolecular NMR Analysis of the Peptide-Oleate Complexes

To define the structural prerequisite for this shared activity, the change in peptide structure after binding to oleate was investigated by NMR spectroscopy. Native protein structure is often regarded as a prerequisite for biological function. When proteins become misfolded, they lose biological activity and may form amorphous aggregates and amyloid fibrils with toxicity for host tissues. In the case of HAMLET, however, we have shown that tumoricidal activity is present when the protein is partially unfolded, in a molten globule-like state. Molten globules retain secondary structural elements but lack tight packing of the interior, resulting in a loss of overall tertiary structure. The polypeptide backbone chain and the side chains of such proteins are in conformational exchange, resulting in broad peaks and poor chemical shift dispersion, as observed by biomolecular NMR spectroscopy (FIG. 10).

The ¹H NMR spectra detected a shift from sharp signals for the naked alpha1- and sar1alpha-peptides (FIGS. 10A and 10B; Black traces), to broad signals and poor chemical shift dispersion (FIGS. 10A and 10B; grey traces), suggesting that the complexes are undergoing conformational exchange on the millisecond time scale. Such broadening is detected in the amide, side chain methyl and aromatic regions, suggesting that interactions between the fatty acid and the respective peptides may be occurring throughout the molecules. A particularly striking example is found in the sar1alpha peptide's Trp indole protons, at approximately 10.2 ppm (FIG. 10B; Black and grey traces). A single, sharp proton signal for the naked sar1alpha peptide (corresponding to each single indole proton present for the three Trp residues in the peptide), has partitioned into two relatively broad signals in the sar1alpha-oleate complex, suggesting that structural changes have yielded different side chain environments (FIG. 10B, black arrow and grey arrow, respectively).

2D NOESY spectral data was acquired and the important NOEs were identified between the olefinic protons (5.23 ppm) of oleic acid and the Hα protons and aromatic protons of the alpha1 peptide (FIG. 10C). Similarly, NOE interactions between the sar1alpha peptide's aromatic region and the oleic acid olefinic protons are clearly observed (FIG. 10D). These spectra clearly show that there existed non-covalent, relatively short through-space interactions (usually less than 5.5 Å to give rise to an NOE signal) between the respective peptides and the fatty acid moieties. The downfield chemical shift of amide protons in alpha1 and the alpha1-oleate complex between 7.6 to 8.8 ppm suggests the presence of secondary structure, corroborating CD spectropolarimetric results (FIG. 1B). Due to the inherent quantitative nature, well-resolved signals obtained from the 1-dimensional ¹H NMR spectra provided a stoichiometry of 3.7 OA molecules for each alpha1 peptide in the complex.

Further evidence of peptide-fatty acid interactions were obtained from chemical shift mapping (CSM) or chemical shift perturbation (CSP), a technique that is widely used to provide binding information on protein-ligand interactions, by comparing peaks that result from the specific environment provided for the C—H bonds present in the protein or peptide (Williamson, 2013). The two-dimensional ¹-¹³C HSQC spectra of alpha1-oleate show a chemical shift perturbation with the aromatic side chain region and the imidazole ring protons compared to the naked peptide (FIG. 10E). These perturbations are also found in the aliphatic region (FIG. 10F), where side chain C—H correlations for the Met Cϵ, the Ile Cδ and Cγ, the Leu Cδ and the Ala Cβ groups are identified to change upon binding of the fatty acid. Based upon these extensive interactions observed with NMR, we conclude that fatty acid binding substantially alters the alpha1- and sar1alpha-peptide structure.

### Example 11

### Free Energy Surface Analyses of the Peptide- and Peptide-Oleate System

The structural heterogeneity was further supported, by molecular modeling, using the Halmiltonian replica exchange molecular dynamics method. By dihedral Principal Component Analysis (Dpca) (Mu et al., 2005), which uses the backbone dihedral angles as internal coordinates for a representation of the dynamics of a peptide, alpha1 and alpha1-oleate were predicted to be located in different regions of the Principal Component subspace, suggesting that they belong to different conformational ensembles (FIGS. 11A-11B).

By assessing the free energy surface, representative structures at each local minima were mapped and ensembles were clustered using the Gromos algorithm (FIG. 11). Local minimum A1 for the alpha1-oleate system revealed a well-defined helices and a hydrophobic oleate lipid core upon which the alpha1 peptide folds in a manner fundamentally different from that of the alpha1 peptide alone (FIGS. 11A-11B). In contrast, multiple local minima (Minima A2, B2, C2, and D2) in naked alpha1 peptide system, were characterized by a relatively less defined secondary structure and various partially folded helix-turn conformations, suggesting the more dynamic nature of the alpha1 peptide in agreement with the NMR structural data (FIGS. 11A and 11B; Black traces). Calculated residue alpha helical propensities of the alpha1-oleate system from simulation revealed defined continuous helical segments of propensities approximately greater than 0.5, however this is not observed for the naked alpha1 system.

Molecular dynamics simulations and the generation of structural ensembles followed by free energy surface analyses of the sar1alpha and the sar1alpha-oleate also resulted in similar prediction of a structural transition from the naked peptide to the complex (FIGS. 11C-11D). In fact, from the CD spectropolarimetry experiments, the transition could be considered more dramatic, as the naked sar1alpha peptide does not possess any noticeable alpha-helical secondary structure but becomes clearly alpha-helical when bound with oleate (FIG. 6B). This finding is also observed in simulation, where the average peptide helical propensity of the naked sar1alpha peptide is significantly lower than that of the sar1alpha-oleate complex. The free energy surface of the sar1alpha-oleate complex contains 2 minima basins A3 and B3 (with the A3 basin harboring the major structural ensemble) (FIG. 9A), and is characterized by containing a prominent alpha-helical secondary structural element, as shown from simulation calculated alpha helical propensities. By contrast, the free energy surface of the naked sar1alpha shows large structural heterogeneity with minima basins A4 represented by random coil structures, minima C4 represented by helical structures, and B4 and D4 represented by beta structures (FIG. 9B).

By contacts probability analysis, the interactions between alpha1 and oleate were mainly hydrophobic, most likely due to the long aliphatic chain structure of oleate. Side chain protons with a contact probability of more than 0.9 with olefinic protons include the aliphatic side chains of Leu8, Leu11, Leu12, Ile15, Leu23, Leu26, and Met30, and agree perfectly with the NOE data from the NOESY spectra (FIG. 10C). Similar findings are observed for the interactions between sar1alpha and oleate. Residues side chain protons with a contact probability of more than 0.9 with olefinic protons include the aliphatic side chains of Ile6, Leu14, Leu17, and Leu19. We speculate that the interaction of oleate with the side chains of these residues could aid in stabilizing the formation of an oleic acid hydrophobic core. The calculated contact probabilities for aromatic protons with that of olefinic protons shows high values for both the alpha1- and sar1alpha-oleate systems, demonstrating a strong correlation between simulation data and NMR data.

Based on these extensive investigations using NMR spectroscopy, CD spectropolarimetry and computational simulations and the strong agreement observed with the experimental aspects and the simulated predicted ensembles, it was clear that completely non-homologous peptides form complexes with shared structural characteristics when bound to oleic acid, which in turn lead to similar cell biological and physiological effects on tumor cells.

### Example 12

### Endophilin Peptide

The method of Example 8 was repeated using peptide comprising residues 1-35 of the human endophilin peptide of SEQ ID NO 5. The complex produced using the peptide was tested alongside the alpha-1 complex described above. The two cell death assays, ATPlite and Prestoblue were used, and viability was also quantified by trypan blue exclusion.

The results are shown in FIG. 12. These show that the complex made with SEQ ID NO 5 has the same qualitative effect as the alpha-1 peptide, albeit with a slightly lower potency.

### Example 13

### Effects with Other Peptides

The method of Example 8 was repeated again, using peptides of SEQ ID Nos 6, 9, 10 and 11. The complexes produced using the peptides were tested alongside the alpha-1 complex described above, and a negative control comprising 175 μM oleate alone, again using the cell death assays, ATPlite and Prestoblue, also by trypan blue exclusion.

The results are shown in FIG. 13. These again illustrate that cell death occurs with each of the peptides tested in a dose dependent fashion.

1. A biologically active complex comprising a peptide of up to 50 amino acids in length which comprises an alpha-helical domain of a protein which has membrane perturbing activity or a variant thereof which lacks cysteine residues, and oleic acid or a salt thereof, provided the protein is other than alpha-lactalbumin.

2. A biologically active complex according to claim 1 wherein the peptide is from 20-40 amino acids in length.

3. A biologically active complex according to claim 1 wherein the protein is coat complex protein such as COPI, COPII, HOPS/CORVET, SEA (Seh1-associated), a clathrin complex, a BAR domain protein such as an endophilin; or a ESCRT complex protein such as Snf7 domain subunits.

4. A biologically active complex according to claim 3 wherein the protein is a COPII family protein such as SAR1.

5. A biologically active complex according to claim 4 wherein the peptide is a peptide of SEQ ID NO 4
MAGWDIFGWF RDVLASLGLW NKH (SEQ ID NO 4).

6. A biologically active complex according to claim 1 where the peptide is a peptide of any one of SEQ ID Nos 5-11.

7. A biologically active complex according to claim 1 which comprises a water soluble oleate salt.

8. A biologically active complex according to claim 7 wherein the salt is an alkali metal salt such as a sodium- or potassium oleate.

9. A method for preparing a biologically active complex according to claim 1 which comprises combining together the peptide with oleic acid or a salt thereof, under conditions in which they form a biologically active complex.

10. A kit comprising a peptide as defined in claim 11 and oleic acid or a salt thereof.

11. A peptide of any one of SEQ ID NOs 4-11.

12. A peptide according to claim 11 which is of SEQ ID NO 4, 5, 6, 9, 10 or 11.

13. A peptide according to claim 12 which is of SEQ ID NO 4.

14. A pharmaceutical composition comprising a biologically acceptable complex according to claim 1 in combination with a pharmaceutically acceptable carrier or excipient.

15. A method for treating cancer which comprises administering to a patient in need thereof, an effective amount of a biologically active complex according to claim 1.

16. A method according to claim 15 wherein the cancer is a human skin papilloma, human bladder cancer, kidney cancer, lung cancer and glioblastomas.

17. A biologically active complex as defined in claim 1 for use in therapy, in particular in the treatment of cancer.


SEQUENCE LISTING (11 sequences)

SEQ ID NO: 1 - protein, 38 aa, Homo sapiens
  Synthetic peptide
KQFTKAELSQLLKDIDGYGGIALPELIATMFHTSGYDT

SEQ ID NO: 2 - protein, 44 aa, Homo sapiens
  Synthetic peptide
IVENNESTEYGLFQISNKLWAKSSQVPQSRNIADISADKFLDDD

SEQ ID NO: 3 - protein, 43 aa, Homo sapiens
  Synthetic peptide
LDDDITDDIMAAKKILDIKGIDYWLAHKALATEKLEQWLAEKL

SEQ ID NO: 4 - protein, 23 aa, Homo sapiens
  Synthetic peptide
MAGWDIFGWFRDVLASLGLWNKH

SEQ ID NO: 5 - protein, 35 aa, Homo sapiens
  Synthetic peptide
MSVAGLKKQFHKATQKVSEKVGGAEGTKLDDDFKE

SEQ ID NO: 6 - protein, 30 aa, Homo sapiens
  Synthetic peptide
SFSSLGLWASGLILVLGFLKLIHLLLRRQT

SEQ ID NO: 7 - protein, 29 aa, Homo sapiens
  Synthetic peptide
SEKKKTRRCNGFKMFLAALSFSYIAKALG

SEQ ID NO: 8 - protein, 30 aa, Homo sapiens
  Synthetic peptide
GTPEYVKFARQLAGGLQCLMWVAAAICLIA

SEQ ID NO: 9 - protein, 29 aa, Homo sapiens
  Synthetic peptide
VQIPYEVTLWILLASLAKIGFHLYHRLPG

SEQ ID NO: 10 - protein, 29 aa, Artificial Sequence
  Synthetic peptide
SEKKKTRRANGFKMFLAALSFSYIAKALG

SEQ ID NO: 11 - protein, 30 aa, Artificial Sequence
  Synthetic peptide
GTPEYVKFARQLAGGLQALMWVAAAIALIA